SEQUENCE LISTING (12 sequences)

SEQ ID NO: 1 - DNA, 24 bp, homo sapien
tgggaggcttgaatactgcctcaa

SEQ ID NO: 2 - DNA, 24 bp, homo sapiens
tctcatagatggtcaatgcggcgt

SEQ ID NO: 3 - DNA, 24 bp, Homo sapiens
aaaccagaggcagaggaagagcaa

SEQ ID NO: 4 - DNA, 24 bp, Homo sapiens
tcgtcccatgtctgaaggcgtaaa

SEQ ID NO: 5 - DNA, 24 bp, Homo sapiens
aggcaccatgggaagtgattcaga

SEQ ID NO: 6 - DNA, 24 bp, Homo sapiens
atttggtaaggcctgagctggagt

SEQ ID NO: 7 - DNA, 24 bp, Homo sapiens
agagctcaggcagaggtttggatt

SEQ ID NO: 8 - DNA, 24 bp, Homo sapiens
acgtggagaagcaagaccagaagt

SEQ ID NO: 9 - DNA, 24 bp, Homo sapiens
agaaagtggcaggccctctttgta

SEQ ID NO: 10 - DNA, 24 bp, Homo sapiens
tgtcctggaagagaaggcaatggt

SEQ ID NO: 11 - DNA, 24 bp, Homo sapiens
accaactgggacgacatggagaaa

SEQ ID NO: 12 - DNA, 24 bp, Homo sapiens
tagcacagcctggatagcaacgta

(19) United States
(12) Patent Application Publication
(10) Pub. No.: US 2012/0009130 A1
(43) Pub. Date: Jan. 12, 2012

(54) Viral Therapy and Prophylaxis Using Nanotechnology Delivery Techniques

(75) Inventors: Krishnan Chakravarthy, Williamsville, NY (US); Suryaprakash Sambhara, Atlanta, GA (US); Paul Knight, Grand Island, NY (US)

(73) Assignee: NANOAXIS, Williamsville, NY (US)

(21) Appl. No.: 13/102,808

(22) Filed: May 6, 2011

Related U.S. Application Data
(60) Provisional application No. 61/331,945, filed on May 6, 2010.

(51) Int. Cl. A61K 31/713; A61P 31/12; C07H 21/02; A61K 9/12; B82Y 5/00

(52) U.S. Cl. 424/43; 536/24.5; 536/23.1; 514/44 A; 977/774

(57) ABSTRACT

The present disclosure provides compositions and methods of enhancing resistance to viral infections through targeted delivery of 5′PPP negative stranded siRNA via nanoparticles, specifically gold nanorods. The 5′PPP activates type I interferon through the signaling cascade providing a novel therapeutic and prophylactic for seasonal and pandemic influenza. The technology described herein also extends the findings to the use of nanoparticles for delivery of genetic material including but not limited to siRNA and microRNA to accomplish targeted nanoparticle based gene therapy.

### CROSS REFERENCED TO RELATED APPLICATION

This application claims the benefit of U.S. Provisional Application No. 61/331,945, filed May 6, 2010, and entitled “Gold Nanorod Delivery of an ssRNA Immune Activator inhibits Pandemic H1N1 Influenza Viral Replication, which is incorporated by reference herein in its entirety.

### FIELD

This application relates to the field of resistance to viral infection. More specifically, this application concerns the delivery of RNA via nanoparticles that enhance viral resistance, and shut down key pathogenic proteins.

### BACKGROUND

The innate immune system is a host's first line of defense against a variety of pathogens. A major mechanism for rapid initiation of host innate immune responses is to recognize conserved motifs or pathogen-associated molecule patterns (PAMPs) unique to pathogens by pattern recognition receptors (PRRs), such as Toll-like receptors (TLRs). Upon recognition of PAMPs, pattern recognition receptors activate signaling pathways that lead to secretion of proinflammatory cytokines, such as type I interferon (IFN-I) that are essential in antiviral immunity. IFN-I can be induced by binding of a variety of pathogen constituents or by products of infection, such as for example intracellular double-stranded RNA (dsRNA), extracellular dsRNA, lipopolysaccharide, single-stranded RNA (ssRNA), and unmethylated CpG DNA.

Several human viruses, including hepatitis C virus (HCV), vaccinia virus, Ebola virus, and influenza virus, have evolved strategies to target and inhibit distinct steps in the early signaling events that lead to IFN-I induction, indicating importance of IFN-I in the host's antiviral response. Additionally, the sequestering of viral dsRNA by nonstructural protein 1 (NS1) of influenza A virus (IAV) during virus replication prevents access of host dsRNA sensors, limiting induction of IFN-I. A role of NS1 of IAV as an IFN antagonist is evidenced by hyper-induction of IFN-I in response to IAV lacking the NS1 gene (delNSl virus) as compared to wild type virus infection. Additionally, ectopic expression of NS1 inhibits activation of IRF-3.

The need exists for compositions that confer protective immunity against viral infection, by circumventing ability of viruses to inhibit IFN-I induction.

### SUMMARY

The following presents a simplified summary to provide a basic understanding of some aspects described herein. This summary is not an extensive overview of the disclosed subject matter. It is not intended to identify key or critical elements of the disclosed subject matter, or delineate the scope of the subject disclosure. Its sole purpose is to present some concepts of the disclosed subject matter in a simplified form as a prelude to the more detailed description presented later.

To address some of the deficiencies associated with conventional techniques and compounds associated with conferring protective immunity against viral infection the subject disclosure describes novel nanomaterial compositions and methods useful for stimulating innate immunity that facilitate inhibiting viral infection as well as enhancing immune responses to vaccines.

Methods of inhibiting viral infection (such as viral infection from an RNA virus for example an ssRNA virus such as influenza virus) are disclosed. These methods include identifying a viral infection to be inhibited and administering an effective amount of a nanoparticle complexed with RNA that stimulates antiviral response and suppresses any pathogen constituents. These methods identifying a viral infection to be inhibited and administering an effective amount of nanoparticle complexed with a RNA with or without 5′PPP end and 19-23 nucleotide signal interference RNA sequence to key viral pathogenic proteins.

### DETAILED DESCRIPTION OF SEVERAL EMBODIMENTS

### Abbreviations

GNR: Gold Nanorods

GNP: Gold Nanoparticles

HCV: hepatitis C virus

IAV: Influenza A virus

IFN-β: interferon-β

IFN-I: Type I interferon

IPS-1: IFN-1 promoter stimulator 1

NS1: nonstructural protein 1

PRR: Pathogen Recognition Receptors

PAMP: Pathogen Associated Molecular Patterns

ssRNA: single-stranded RNA

siRNA: signal-interference RNA

### Terms

Unless otherwise noted, technical terms are used according to conventional usage.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.” In case of conflict, the present specification, including explanations of terms, will control. In addition, all the materials, methods, and examples are illustrative and not intended to be limiting.

To facilitate review of the various embodiments of the disclosure, the following explanations of specific terms are provided:

A “nanoplex” is any nanoparticle complexed to any siRNA, nucleotide sequence or genetic material.

A “host cell” is a cell which has been transformed, or is capable of transformation, by an exogenous nucleic acid sequence, such as 5′PPP-ssRNA or ssRNA. A cell has been “transformed” by exogenous nucleic acid when such exogenous nucleic acid has been introduced inside the cell membrane.

“Nucleotide” includes, but is not limited to, a monomer that includes a base linked to a sugar, such as a pyrimidine, purine or synthetic analogs thereof, or a base linked to an amino acid, as in a peptide nucleic acid (PNA). A nucleotide is one monomer in a polynucleotide. A nucleotide sequence refers to the sequence of bases in a polynucleotide. For example, a RIG-I polynucleotide is a nucleic acid encoding a RIG-I polypeptide.

Conventional notation is used herein to describe nucleotide sequences: the left-hand end of a single-stranded nucleotide sequence is the 5′-end; the left-hand direction of a double-stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand;” sequences on the DNA strand having the same sequence as an mRNA transcribed from that DNA and which are located 5′ to the 5′-end of the RNA transcript are referred to as “upstream sequences;” sequences on the DNA strand having the same sequence as the RNA and which are 3′ to the 3′ end of the coding RNA transcript are referred to as “downstream sequences.”

Pharmaceutical agent: A chemical compound or composition capable of inducing a desired therapeutic or prophylactic effect when properly administered to a subject or a cell. “Incubating” includes a sufficient amount of time for interaction with a cell. “Contacting” is placement in direct physical association. Includes both in solid and liquid form. Contacting can occur in vitro with isolated cells or in vivo by administering to a subject. “Administrating” to a subject includes topical, parenteral, oral, intravenous, intra-muscular, sub-cutaneous, inhalational, nasal, intra-articular or dermal administration, among others.

An “anti-viral agent” is an agent that specifically inhibits a virus from replicating or infecting cells.

A “therapeutically effective amount” is a quantity of a chemical composition or an anti-viral agent sufficient to achieve a desired effect in a subject being treated. For instance, this can be the amount necessary to inhibit viral replication or to measurably alter outward symptoms of the viral infection, such as a decrease or lack of symptoms associated with a viral infection. In general, this amount will be sufficient to measurably inhibit virus replication or infectivity. When administered to a subject, a dosage will generally be used that will achieve target tissue concentrations that has been shown to achieve in vitro inhibition of viral replication.

Pharmaceutically acceptable carriers: The pharmaceutically acceptable carriers of use are conventional. Remington's Pharmaceutical Sciences, by E. W. Martin, Mack Publishing Co, Easton, Pa., 15th Edition, 1975, describes compositions and formulations suitable for pharmaceutical delivery of the compositions disclosed herein. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Polypeptide: Any chain of amino acids, regardless of length or post-translational modification (such as glycosylation or phosphorylation). “Polypeptide” applies to naturally occurring amino acid polymers and non-naturally occurring amino acid polymers as well as polymers in which one or more amino acid residue is a non-natural amino acid, for example an artificial chemical mimetic of a corresponding naturally occurring amino acid. A “residue” refers to an amino acid or amino acid mimetic incorporated in a polypeptide by an amide bond or amide bond mimetic. A polypeptide has an amino terminal (N-terminal) end and a carboxy terminal (C-terminal) end. “Polypeptide” is used interchangeably with peptide or protein, and is used interchangeably herein to refer to a polymer of amino acid residues.

Preventing, Inhibiting or Treating a Disease: Inhibiting full development of a disease or condition, for example, in a subject who is at risk for a disease such as viral infection, for example an infection with an RNA virus, a dsRNA virus, or a ssRNA virus such as an influenza virus. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease. A “prophylactic” treatment is a treatment administered to a subject who does not exhibit signs of a disease or exhibits only early signs for the purpose of decreasing the risk of developing pathology. A “prophylactic” includes vaccination against the disease or condition, for example, vaccination against a viral infection.

Purified: The term “purified” (for example, with respect to a nanoparticle complex or negative stranded RNA) does not require absolute purity; rather, it is intended as a relative term. Thus, for example, a purified nucleic acid is one in which the nucleic acid is more enriched than the nucleic acid in its natural environment within a cell. Similarly, a purified peptide preparation is one in which the peptide or protein is more enriched than the peptide or protein is in its natural environment within a cell. In one embodiment, a preparation is purified such that the specified component represents at least 50% (such as, but not limited to, 70%, 80%, 90%, 95%, 98% or 99%) of the total preparation by weight or volume.

Vaccine: A vaccine is a pharmaceutical composition that elicits a prophylactic or therapeutic immune response in a subject. In some cases, the immune response is a protective immune response. Typically, a vaccine elicits an antigenspecific immune response to an antigen of a pathogen, for example to a virus. The vaccines described herein include nanoplex compositions or nanoparticles complexed with negative stranded RNA.

Virus: Microscopic infectious organism that reproduces inside living cells. A virus consists essentially of a core of nucleic acid surrounded by a protein coat, and has the ability to replicate only inside a living cell, for example as a viral infection. “Viral replication” is the production of additional virus by the occurrence of at least one viral life cycle. A virus, for example during a viral infection, may subvert the host cells' normal functions, causing the cell to behave in a manner determined by the virus. For example, a viral infection may result in a cell producing a cytokine, or responding to a cytokine, when the uninfected cell does not normally do so.

An “RNA virus” is a virus which belongs to either Group III, Group IV or Group V of the Baltimore classification system (see, Luria, et al. General Virology, 3rd Edn. John Wiley & Sons, New York, p2 of 578, 1978). RNA viruses possess ribonucleic acid (RNA) as their genetic material and typically do not replicate using a DNA intermediate. The nucleic acid is usually single-stranded RNA (ssRNA) but can occasionally be double-stranded RNA (dsRNA). Group III viruses include dsRNA viruses, for example viruses from: Birnaviridae, Chrysoviridae, Cystoviridae, Hypoviridae, Partitiviridae, Reoviridae (such as Rotavirus), and Totiviridae among others. Group IV includes the positive sense ssRNA viruses and includes for example viruses from: Nidovirales, Arteriviridae, Coronaviridae (such as Coronavirus and SARS), Roniviridae, Astroviridae, Barnaviridae, Bromoviridae, Caliciviridae, Closteroviridae, Comoviridae, Dicistroviridae, Flaviviridae (such as Yellow fever virus, West Nile virus, Hepatitis C virus, and Dengue fever virus), Flexiviridae, Hepeviridae (such as Hepatitis E virus), Leviviridae, Luteoviridae, Marnaviridae, Narnaviridae, Nodaviridae Picornaviridae (such as Poliovirus, the common cold virus, and Hepatitis A virus), Potyviridae, Sequiviridae, Tetraviridae, Togaviridae (such as Rubella virus and Ross River virus), Tombusviridae, and Tymoviridae among others. Group V viruses are negative sense ssRNA viruses and include for example viruses from: Bornaviridae (such as Borna disease virus), Filoviridae (such as Ebola virus and Marburg virus, Paramyxoviridae (such as Measles virus, and Mumps virus), Rhabdoviridae (such as Rabies virus), Arenaviridae (such as Lassa fever virus), Bunyaviridae (such as Hantavirus), and Orthomyxoviridae (such as Influenza viruses) among others.

“Influenza viruses” have a segmented single-stranded (negative or antisense) genome. The influenza viron consists of an internal ribonucleoprotein core containing the single-stranded RNA genome and an outer lipoprotein envelope lined by a matrix protein. The segmented genome of influenza A consists of eight linear RNA molecules that encode ten polypeptides. Two of the polypeptides, HA and NA, include the primary antigenic determinants or epitopes required for a protective immune response against influenza. Based on the antigenic characteristics of the HA and NA proteins, influenza strains are classified into subtypes. “Avian influenza” usually refers to influenza A viruses found chiefly in birds. Recent outbreaks of avian influenza in Asia have been categorized as H5N1, N7N7 and H9N2 based on their HA and NA phenotypes. These subtypes have proven highly infectious in poultry and have been able to jump the species barrier to directly infect humans causing significant morbidity and mortality.

### BRIEF DESCRIPTION OF THE DRAWINGS

FIG. 1 depicts measurement of GNR-ssRNA complex formation and binding efficiency.

FIG. 1A depicts localized longitudinal surface plasmon resonance peak of GNRs red shift upon complex formation with RNAs.

FIG. 1B depicts lanes loaded with 900 ng 5′PPP-ssRNA, either alone (lanes 1, 3, 5, and 7) or after premixing with 250 ng GNR (lanes 2 and 8), 500 ng GNR (lanes 4 and 9), or 750 ng GNR (lanes 6 and 10). Lanes 1-6 were visualized by ethidium bromide staining under UV light and lanes 7-10 were visualized with white light.

FIG. 2 depicts uptake of GNR-5′PPPssRNA by A549 cells. Cellular uptake and internalization of nanoplexes (GNR-5′PPPssRNA) in A549 cells has been visualized using transmission electron microscopy. GNR-5′PPPssRNA nanoplexes are clearly visible in endocytic compartments within the cell (indicated by arrows in A through D).

The analysis of FIG. 3 depicts 5′PPP-ssRNA induced expression of RIG-I and IFN-β in A549 cells. A549 (3.5×105 cells/well) in a 6-well tissue culture plate were mock-transfected (control) or transfected with 3 μg/ml of RNA complexed with 2.5 μg of GNR. GNR-5′PPP, GNRCapped, and GNR-CIAP were used.

FIG. 3A depicts IFN-β after 24, 48, and 72 hours of treatment and

FIG. 3B shows RIG-I expression after 24, 48, and 72 hours, each of IFN-β and RIG-I were analyzed by quantitative RT-PCR. All data were normalized to β-actin, a housekeeping gene and expressed as fold increases. Data shown represent the mean±SD of three independent experiments and p values are given for the comparison of GNR-5′PPP with GNR alone.

FIG. 3C A549 cells were transfected as mentioned in IFN-β, RIG-I and 48 and 72 hours post-transfection RIG-I protein expression was analyzed by immunoblot.

FIG. 4 depicts GNR-5′PPP-ssRNA enhances IFN-β, RIG-I, and MDA5 expression, and inhibits NS1 expression following infection with 2009 pandemic H₁N₁ influenza viruses and Solomon Islands seasonal flu strain. A459 cells (3.5×105 cells/well) in a 6-well tissue culture plate were mock-transfected or transfected with 3 μg of RNA's complexed with 2.5 μg of GNR per well for 48 hours and then infected with A/California/08/09 or A/Solomon Islands/03/06 at an MOI of 1. Lysates to determine mRNA levels by QRTPCR (FIGS. 4A and 4B) and protein to assess the levels of NS1, RIG-I, MDA5, and IPS-1, (FIGS. 4C and 4D) were collected 24 hours later. Data shown in A, B, and C are for cultures infected with A/California/08/09 and the data shown in D are for A/Solomon Islands/03/06. Results shown in FIG. 4A and FIG. 4B are mean±SD from two independent experiments.

FIG. 5 depicts GNR-5′PPP-ssRNA inhibits replication of 2009 pandemic H1N1 influenza viruses and Solomon Islands seasonal flu strain. A459 cells (3.5×105 cells/well) in a 6-well tissue culture plate were mock-transfected or transfected with 3 μg of RNAs complexed with 2.5 μg of GNR per well for 48 hours and then infected with A/California/08/09 or A/Solomon Islands/03/06 at an MOI of 1. The viral titers were determined from the supernatants collected 24 hours later. Results shown are mean±SD from two independent experiments and are expressed as viral titer (pfu/ml).

FIG. 6 illustrates study of nanoplex distribution in A459 cells. Dark-field and fluorescence images were acquired on cells following treatment with GNR-siRNAF nanoplex, free siRNAF (negative control), and siPORT-siRNAF (positive control). Fluorescence images show robust uptake of the GNR-siRNAF and siPORT-siRNAF as opposed to free siRNAF. The dark-field images of GNRs corresponding to the longitudinal surface plasmonic enhancement in the red region can be clearly visualized in the samples treated with GNR-siRNAF.

FIG. 7 illustrates fluorescence spectra of siRNAF from A549 lysates. Data show the highest values of fluorescence intensity in the samples treated with GNR-siRNAF as compared with samples transfected with siRNAF alone or siPORT-siRNAF.

FIG. 8 illustrates cell viability (MTT) assay of A459 cells following treatment with GNR, GNR-Capped, and GNR-5′PPP-ssRNA nanoplexes. Results show minimal toxic effects on the cells following treatment with the nanoplexes, which were observed up to 96 hour posttreatment. The results are the mean±SD of three separate experiments.

FIG. 9 illustrates RT-PCR-Array analysis of IFN-stimulated genes. A459 cells were mock transfected or transfected with 3 μg of RNA complexed with 2.5 μg of GNR per well for 48 h and then either mock infected or infected with A/California/08/09 at an MOI of 1 for 24 h. Columns represent the fold differences in the mRNA levels of selected IFN-stimulated genes compared to the mock-transfected and uninfected controls.

FIG. 10 illustrates quantification of secreted IFN-β. A459 cells were mock transfected or transfected with 3 μg of RNA complexed with 2.5 μg of GNR per well for 48 h and

FIG. 11 illustrates cells infected with A/California/08/09 or A/Solomon Islands/03/06 at an MOI of 1 for 24 h. Secreted IFN-β levels in cell culture supernatants were determined by ELISA.

All of the findings in the disclosed figures indicate that nanoplex delivery of innate immune activators is sufficient to effectively impair the replication of both seasonal and pandemic H1N1 influenza viruses.

### DESCRIPTION OF SEVERAL EMBODIMENTS

A novel influenza A/H1N1 virus containing genome segments derived from avian, human, and porcine species was first isolated in April 2009 and quickly spread globally prompting the World Health Organization (WHO) to declare a pandemic. As of Oct. 24, 2009, WHO reported at least 414,000 confirmed cases and nearly 5000 deaths globally. Although the actual number of total cases is likely to be many fold higher, since current surveillance is focused only on severe and fatal cases. The United States government has declared the H1N1 pandemic a national emergency with significant impact on public healthcare. Although vaccination programs form the backbone of public-health intervention strategies, lengthy egg-derived H1N1 vaccine production timelines, suboptimal growth of vaccine strain viruses, and limited current manufacturing capacities delayed the availability of pandemic influenza vaccine.

Antiviral drugs are another public health tool for prophylactic and therapeutic interventions against influenza. There are currently two classes of anti-influenza virus drugs: the M2 ion channel blockers (amantadine, rimantadine) and the neuraminidase inhibitors (oseltamivir, zanamavir). However, the emergence of influenza viral strains resistant to both of these classes of antiviral drugs is becoming increasingly common, highlighting the importance of devising new preventive and therapeutic strategies, particularly those that can be delivered effectively to severely ill patients together with appropriate clinical management and the use of lung protective strategies. One recent pharmacological approach has been the development of small molecules to augment the host innate immune response.

The innate immune system has evolved to recognize viral pathogens via the pathogen recognition receptors (PRRs). Recognition of pathogen associated molecular patterns (PAMPs) by PRRs results in rapid induction of anti-viral cytokines, such as IFN-1, as well as cytokines responsible for the formation of adaptive immunity. Influenza viral RNA is detected by the cytosolic RNA sensor RIG-I. Following binding to RNA {double stranded (ds)} or 5′PPP-single stranded (ss)), RIG-I undergoes a conformational change allowing it to interact with IFN-1 promoter stimulator 1 (IPS-1). The interaction of IPS-1 and RIG-I leads to the induction of type I IFN genes and innate immune response cytokines. Hence, activation of RIG-I by its 5′PPPssRNA ligand is an attractive alternative to existing prophylactic treatments.

Also, since innate immunity is evolutionarily conserved and significant for host survival independent of viral strain, viral resistance to this therapeutic approach is less likely to develop. The major problem with using 5′PPP-ssRNA to activate RIG-I is the difficulty in delivering this ligand. In recent years, gold nanoparticles (GNP), gold nanorods (GNR) and nanoparticles in general have gained increasing interest as potential biocompatible and site-specific carriers of various diagnostic and therapeutic agents.

Recently, we have used GNR to deliver siRNA to silence genes that are associated with opiate drug addiction. GNR surfaces can be easily modified to incorporate cationic charges, which facilitate their stable electrostatic interaction with anionic genetic materials making them suitable delivery vehicles. In this disclosure, we show GNR-mediated delivery of ssRNA as a novel therapeutic paradigm for treatment of seasonal and pandemic flu.

Disclosed herein is that GNR enhanced delivery of bioactive 5′PPP-ssRNA RIG-I ligand, results in up-regulation of type I IFN through stimulation of RIG-I. Increased type I IFN production will reduce concomitant viral replication. Results demonstrate the successful internalization of GNR-5′PPP-ssRNA nanoplexes, up-regulation of antiviral responses, and reduction of replication of both a seasonal influenza A virus (A/Solomon Islands/03/06) and a 2009 H1N1 pandemic virus (A/California/08/09). These findings disclose a nanotechnology-based novel approach to stimulate antiviral responses of the host innate immune system.

A: Electrostatic Binding of GNR to 5′PPP-ssRNA

Electrostatic binding of 5′PPP-ssRNA with GNR to form biocompatible nanoplexes to determine successful complex formation of gold nanorods to various nucleic acid constructs we used three different methods: surface plasmon resonance shifts, changes in zeta potential, and gel electrophoresis studies. Production of nanoplexes was accomplished by mixing the cationic GNR substrate with the anionic nucleic acid ligands. Determination of successful complex formation is dependent on two factors.

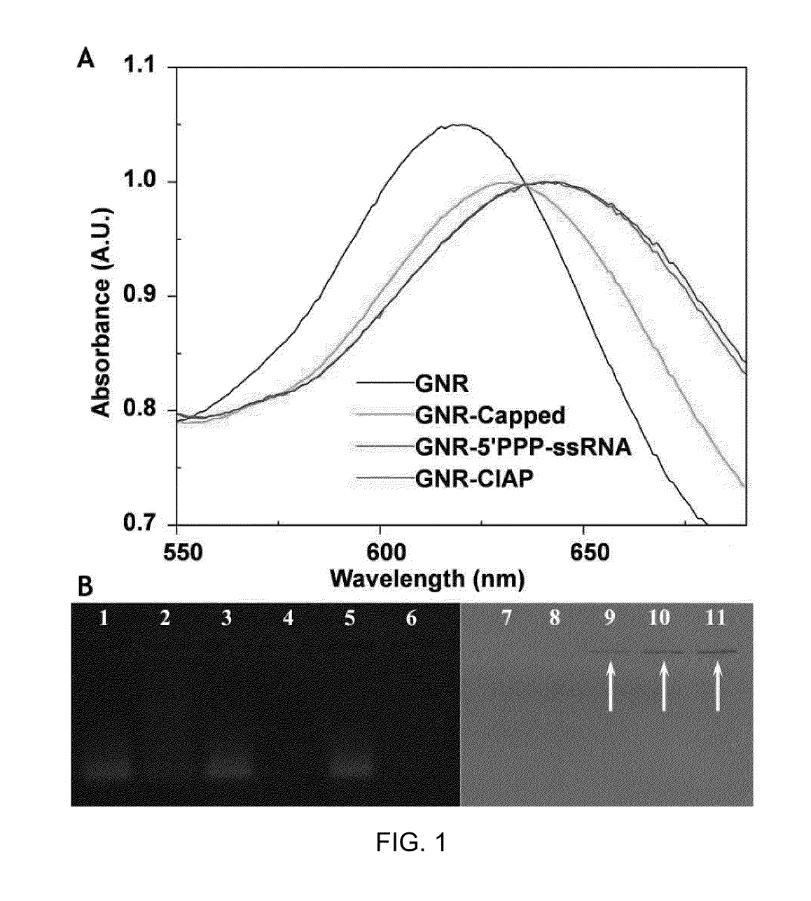

First, efficient complex formation of the GNRs with RNA results in changes in the local refractive indices around the GNRs, resulting in a red shift in the localized longitudinal surface plasmon resonance peak as shown in FIG. 1A. 5′PPP containing ssRNA activates RIG-I mediated antiviral response; however, synthetic RNAs that lack 5′PPP groups fail to activate the RIG-I pathway. Hence, in studies used in vitro transcribed ssRNA that contains 5′PPP moiety and as negative controls, we removed 5′PPP group by treating ssRNA with CIAP or capped 5′PPP group during synthesis so that 5′PPP group is no longer available for RIG-I interaction. We observed a 14 nm shift between GNR alone and GNR upon complex formation with Capped-ssRNA (GNR-Capped). However, with bound 5′PPP-ssRNA (GNR-5′PPP) and CIAP-ssRNA (GNR-CIAP) we observed a 23 nm and 25 nm shift, respectively. Thus, surface plasmon resonance becomes a significant nanotechnological tool to determine if binding had indeed occurred between GNR and RNAs.

Second, binding of RNA on the GNR surface reduces the overall net charge of the nanoplex. We observed that the zeta potential of free GNR is +20.71 mV, and upon successful complex formation to 5′PPP-ssRNA, CIAP-ssRNA, and Capped-ssRNA, it decreased to −9.91 mV, −9.61 mV, and −8.23 mV, respectively (Table S1). These results suggest that binding of cationic GNRs to anionic nucleic acid material leads to a slightly negatively charged nanoplex and that complexing of genetic material to GNR would increase uptake of the nanoplexes into the target cell due to evasion of the reticuloendothelial system and reduction in non-specific interactions with proteins and other biomolecules as demonstrated by other studies.

To identify the amount of GNR needed to completely bind a given amount of ssRNA, we conducted gel electrophoresis studies. Results (FIG. 1B) show that addition of increasing amounts of GNR to a constant amount of 5′PPP-ssRNA leads to a decrease in the amount of free 5′PPP-ssRNA, visible by EtBr staining, and increased nanoplex formation (FIG. 1B, lanes 1-6). Lanes 1, 3, 5 were used as control lanes with free 5′PPP-ssRNA. To verify the presence of the immobile nanoplex in the gel, we visualized the gel under visible light (FIG. 1B, lanes 7-10). Increasing amounts of GNR-5′PPP nanoplex, correlated to the increasing GNR added to the sample, as visualized by the purple lines in the wells marked by the arrows. Thus, based on these electrophoresis studies it was concluded that each μg of GNR preparation can bind approximately 1.2 μg ssRNA. Thus, the combination of plasmonic shift experiments, charge determination, and changes in electrophoresis migration confirmed the successful complex formation between GNR to RNA constructs. GNR nanoplexes are internalized by A549 cells with minimal cytotoxicity. The longitudinal surface plasmon oscillation of the GNRs gives a strong plasmonic scattering in the orange-red region of the optical spectrum. This phenomenon can be used to study the intracellular distribution of nanoplexes by dark field microscopy.

Here we examined the intracellular delivery of GNR conjugated to a fluorescently labeled siRNA (siRNAF) in A459 cells using dark-field imaging FIG. S1 shows the dark-field and fluorescence images of A459 cells, with and without treatment with the GNR-siRNAF nanoplex. Commercial siPORT (Ambion) was employed as a positive control transfection agent. The rate of release of ssRNA species from the GNR either in solution or after transfection into cells could not be determined due to the lack of a sensitive assay to determine the quantity of the ssRNA as it is not fluorescently labeled. Furthermore, free RNA species are degraded by RNAses that are abundant in culture media. The intracellular delivery of the nanoplexes can be easily observed from the strong orange-red light scattering, a property of GNR. Since it is not possible to determine intracellular localization with Dark Field microscopy, we used confocal microscopy using Z-slices as well as TEM which clearly demonstrate the uptake of GNR, perhaps through micropinocytosis. Thus, another advantage of using nanotechnology in the delivery of therapeutics is that the unique properties of the nanoparticles also can be exploited to monitor their cellular entry and distribution.

We also measured fluorescence from cellular lysates following their treatment with either free siRNAF, siRNAF complexed with GNRs, or siRNAF complexed with the commercially available gene-silencing agent siPORT to confirm darkfield images. Results indicate that the fluorescence from lysates of cells treated with GNR-siRNAF is approximately 10% higher than from lysates of cells treated with siPORT-siRNAF indicating that the intracellular delivery efficiency of siRNA using GNRs is as good as commercially available gene silencing agent (FIG. S2).

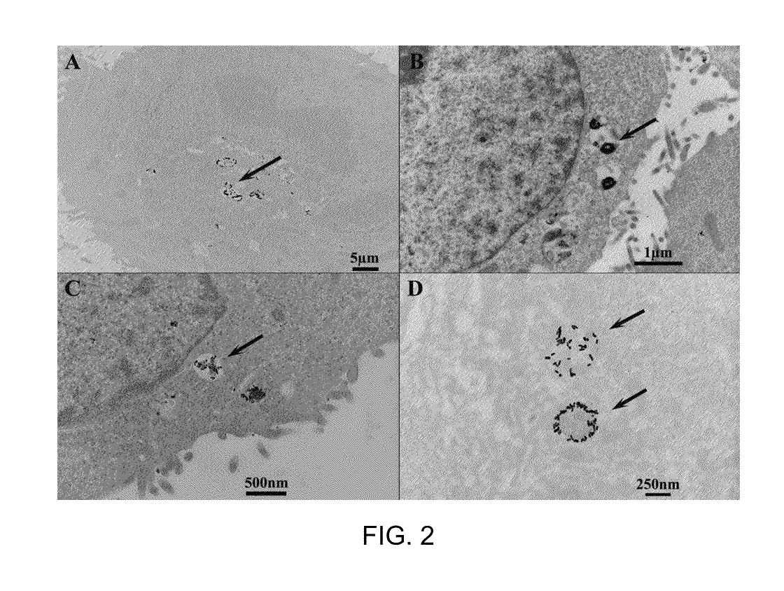

To specifically determine the uptake and intracellular distribution of nanoplexes (GNR-5′PPP-ssRNA) in A549 cells we employed transmission electron microscopy (TEM). Cells were treated with nanoplexes for 24 hours and viewed by TEM. FIG. 2, Panels A to D shows the presence of these nanoplexes in endocytic vesicles. We postulate that the particles may be taken up by classical pinocytotic mechanisms of uptake but further confirmatory studies are required (38, 39).

To determine toxicity associated with uptake of the GNR nanoplexes, a quantitative MTT cell viability assay 24, 48, 72, and 96 hours post transfection was employed. Cell death detected after transfection with GNR, GNR-5′PPP, or GNR-Capped nanoplexes at all time points examined ranged from 0-0.8%, 7.8%-8.8%, and 0.8%-7.7%, respectively (FIG. S3). Induction of IFN-1 and RIG-I expression by GNR-5′PPP-ssRNA

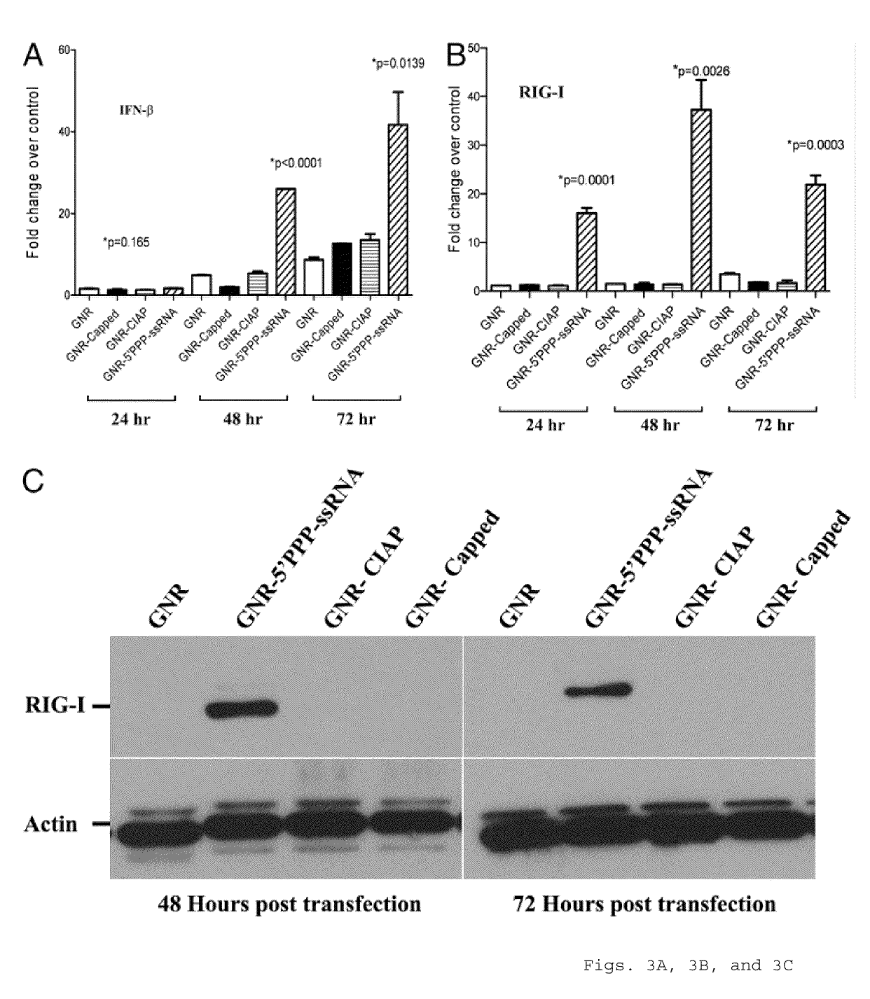

Although the nanoplexes clearly enter the cell (FIG. 2), it was desired to specifically address ability of the RNA ligand to activate innate immune PRRs. Previous laboratory experiments have shown that using cationic lipids to transfect 5′PPPssRNA into A549 cells activated RIG-I and induced IFN-1 expression. To determine whether or not GNR-based nanoplexes could similarly upregulate the type I IFN response, changes were assessed in the message levels of RIG-I and IFN-1 by quantitative RT-PCR. Transcription of IFN-1 increased for at least 72 hours following treatment of A549 cells with the GNR-5′PPPssRNA nanoplexes, reaching a maximum of ˜40-fold above untreated controls (FIG. 3A).

Addition of GNR alone, or GNR conjugated to capped-RNA or

CIAP-RNA led to only marginal increases in IFN-1 message levels. RIG-I expression was also increased by GNR-5′PPP nanoplexes but not by GNR alone, GNR-Capped, or GNR-CIAP nanoplexes (FIG. 3B). Increased RIG-I mRNA was correlated with a corresponding increase in RIG-I protein levels as assessed by Western blot analysis. At 48 hrs or 72 hrs following transfection, strong bands corresponding to RIG-I can clearly be detected in the A459 cells, but not in control cells that received either GNR alone, GNR-Capped, or GNR-CIAP (FIG. 3C). Besides inducing expression of RIG-I and IFN-1, GNR-5′PPP-ssRNA complexes also enhanced the levels of IFN-responsive genes, PKR, MDA5, IRF1, IRF7, MX1, CXCL10, ISG12 and others, while GNR-alone or GNR-Capped had little or no impact on the expression of these genes (FIG. S5).

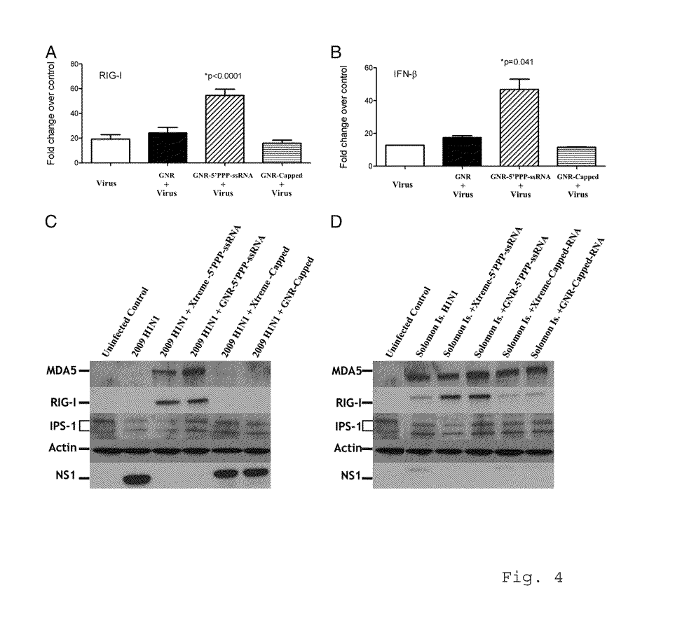

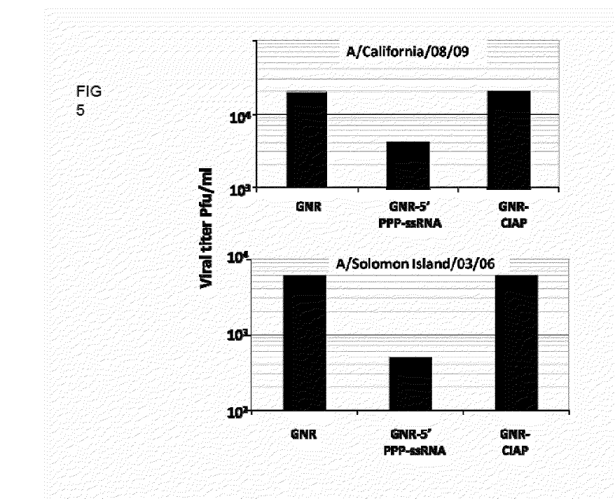

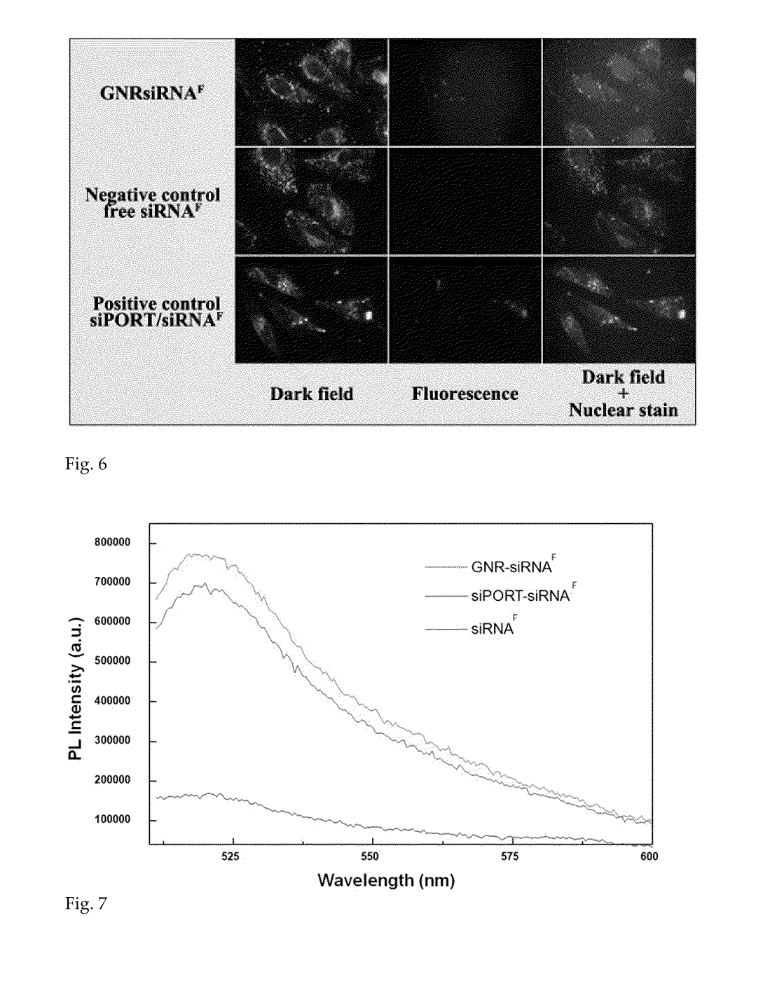

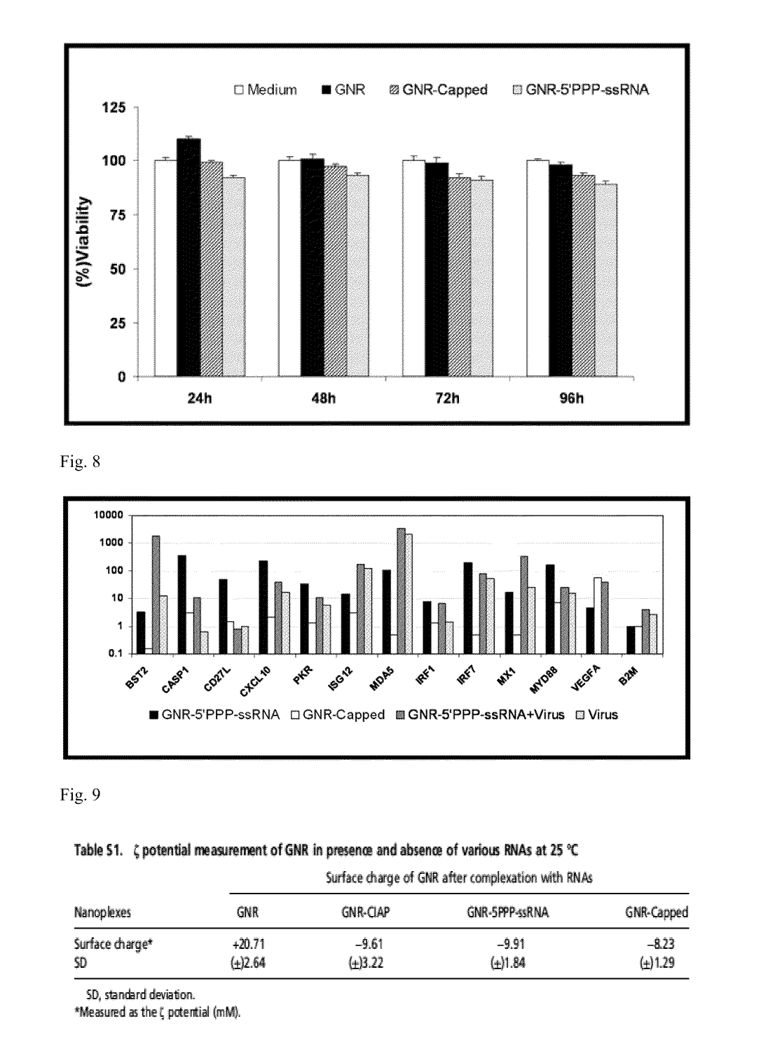

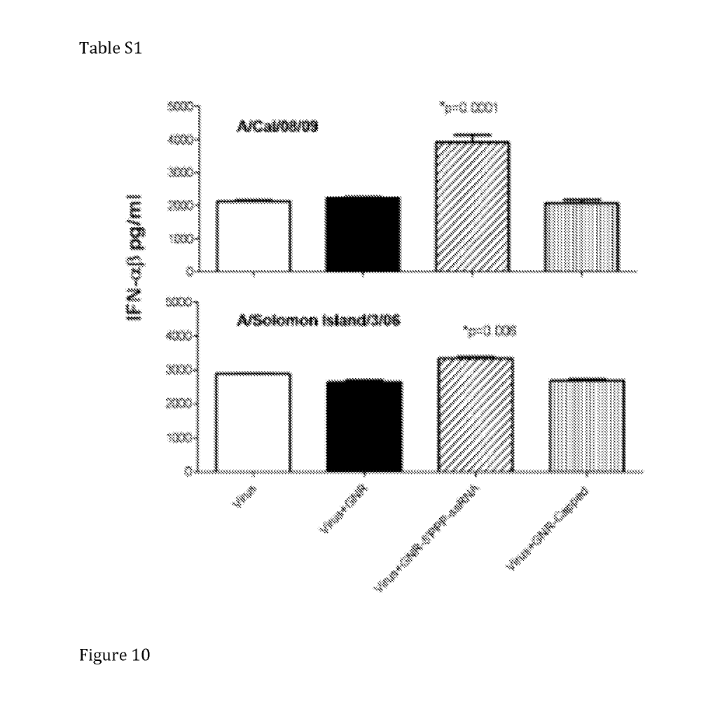

Antiviral bioactivity of GNR-5′PPP-ssRNA: A determination was made whether level of RIG-I activation achieved by treatment with GNR-5′PPP was sufficient to inhibit replication of seasonal (e.g., A/Solomon Islands/03/06) or 2009 H1N1 pandemic (e.g., A/California/08/09) influenza virus strains. To do this, A549 cells were first treated with GNR nanoplexes and then infected with the appropriate influenza virus 48 hours later. Samples were harvested and analyzed 24 hours after viral infection. Infection with A/California/08/09 virus failed to upregulate RIG-I and IFN-1 message (FIGS. 4A, and 4B) or RIG-I protein (FIG. 4C); however, there was significant increase in NS1 expression (FIG. 4C) and viral titers (FIG. 5).

Nevertheless, pretreatment with GNR-5′PPP nanoplexes, but not with GNR-Capped or GNR-CIAP nanoplexes or GNR alone, increased IFN-1 message (FIG. 4B) and protein (FIG. S4) and both RIG-I message and protein levels (FIGS. 4A and 4C) over the levels seen with virus only. Furthermore, the treatment also reduced amounts of NS1 below level of detection (FIG. 4C) and viral titers by approximately 90% (FIG. 5). Similarly, pretreatment with GNR-5′PPP subsequently inhibited induction of NS1 and upregulated RIG-I expression post-infection with a seasonal influenza virus, A/Solomon Islands/03/06 (FIG. 4D). These findings suggest that nanoplex delivery of innate immune activators is sufficient to effectively impair the replication of both seasonal and pandemic H1N1 influenza viruses.

B. Discussion of Delivery Mechanism Benefits:

This research has evaluated use of GNR nanotechnology and nanoparticles in general to deliver 5′PPP-ssRNA, an innate immune activator with antiviral action against influenza virus infections. Gold-based nanoparticles and nanorods have gained increasing interest as a safe delivery system for therapeutic nucleic acids because of their biocompatibility and capacity to form stable nanoplexes. Lungs are especially well suited for this novel therapeutic nanoplex delivery strategy as direct contact with the environment provides a portal for inhalation administration, avoiding parenteral injection. In particular, site-specific delivery of type I IFN or IFN-inducers can potentially reduce systemic side effects, in addition to having a beneficial therapeutic outcome of reducing influenza virus replication. The recent spread of the 2009 H1N1 pandemic influenza viruses, as well as drug resistant seasonal viruses, and the potential threat of highly pathogenic avian influenza viruses have intensified the search for new classes of antiviral drugs and therapeutic strategies.

A limitation of ssRNA therapy is sensitivity of RNA to rapid degradation. Despite some of initial successes in overcoming this ability, most current nucleic acid delivery systems have limitations based on cellular toxicity (e.g., cationic lipid complexes) or untoward immune responses and toxicity (e.g., virus-based systems). Findings clearly demonstrate that GNR complex formation enhances 5′PPP-ssRNA delivery to human bronchial epithelial cells and results in a bio-functional outcome with limited effects on cell viability. Complex formation of nucleic acid to GNR does not inhibit bioactivity of 5′PPP-ssRNA as signaling through RIG-I pathway that triggers induction of type I IFNs is still active following successful delivery of the nanoplex.

RIG-I induced type I interferon activation response is conserved among positive single strand RNA viruses, suggesting that 5′PPP-ssRNA induction of type I IFN can be extended as a treatment modality for these viruses. In addition to inducing secretion of type I IFNs, 5′PPP-ssRNA also results in induction of other innate immune cytokines, which may be significant for recruiting and activating leukocytes to the site of infection for viral clearance initiating a successful adaptive immune response.

In summary, disclosed is a new therapeutic strategy based on nanotechnology enhanced RNA delivery to potentially treat influenza, as well as other viral infections, where type I IFNs are part of a significant pathway to resolution of infection. Findings clearly demonstrate utility of a novel, noncytotoxic, antiviral strategy of employing GNR-5′PPP-ssRNA nanoplexes or any nanoparticle electrostatically bound to 5′PPP-ssRNA that can activate intracellular antiviral signaling pathways in respiratory epithelial cells, and can specifically inhibit both an H1N1 and seasonal strain of influenza virus replication. Since innate immune response pathways are activated, this approach has potential application to prevent and treat diseases caused by other viruses. Furthermore, ability of viruses to develop resistance is remote as these pathways are evolutionarily conserved. This study clearly demonstrates feasibility of employing biocompatible nanoparticle constructs of GNR complexed with specifically selected ligands (e.g., 5′PPP-ssRNA) to target cytosolic receptors that can trigger pathogen recognition pathways (e.g., RIG-I/MDA-5) to control and treat infectious disease.

C. Materials and Methods:

Cell Lines: A549 cells were grown in DMEM (Life Technologies, Grand Island, N.Y.) supplemented with 10% fetal bovine serum (FBS), 100 U/ml penicillin and 100 μg/ml streptomycin. Influenza viruses, Seasonal influenza virus, A/Solomon Islands/03/06 and the pandemic influenza virus, A/California/08/09 used in this study were obtained from the influenza division, CDC repository. Infections of A549 cells were carried out at a multiplicity of infection (MOI) of 1. Each treatment was carried out in duplicate cultures. After 24 hour post infection with viruses, cell-culture supernatants were collected and stored at −80° C. for determination of viral titer by plaque assay as described previously using Madin-Darby Canine Kidney (MDCK) epithelial cells (43).

Preparation of ssRNA: RNAs (5′PPP-ssRNA, Capped-ssRNA, and CIAP-ssRNA) used in this work_were synthesized with MEGAscript T7 High Yield Transcription Kit (Ambion, Austin, Tex.) using a double stranded DNA template made by annealing complementary 7 oligonucleotides. A template was then digested with DNase I (NEB, Ipswitch, Mass.) and the RNA purified with TRIzol reagent (Invitrogen, Carlsbad, Calif.). Capped RNAs were made by substituting a 12:1 ratio of m7G(5′)PPP(5′)G cap analog:GTP for GTP in the transcription reaction. CIAP-ssRNA was made by removing the functional 5′PPP end with calf intestinal alkaline phosphatase (CIAP) treatment. Kits and reagents were used according to manufacturer's protocols. 5′PPP-ssRNA activates RIG-I and as controls the same 5′PPPssRNA from which 5′PPP group is removed enzymatically with CIAP or blocked 5′PPP group during synthesis by capping were employed.

Nanoplex preparation and analysis: GNRs were synthesized as previously described (Ding; Yong; et al. 2007; Bonoiu, Mahajan et al. 2009). Nanoplex formulation was prepared just prior to each experiment by electrostatically attaching 1 ug of cationic GNR to 1.2 ug of the appropriate RNA (5′PPP-ssRNA, CIAP-ssRNA, or Capped-ssRNA) in Opti-MEM medium (Invitrogen) and incubating at room temperature for 5 minutes. Size of the nanoparticles ranged from 35-70 nm as described earlier (Ding; Yong; et al. 2007). Electrophoretic assessment of nanoplex formation was done according to standard procedures (33) using a 1.5% agarose gel in a tris acetate EDTA buffer system. For TEM, transfected cells were fixed as described (26), sectioned (70-100 nm), stained with lead citrate, and viewed with a Tecnai-12 electron microscope (Phillips, Eindhoven, The Netherlands) at 120 kV. Zeta potential measurements of GNR in the presence and absence of RNA molecules were acquired at 25° C. using a 90 Plus particle size analyzer (Brookhaven Instrument Corp., NY, USA).

Bio-functional analysis following viral infections: A549, human respiratory epithelial and Madin Darby canine kidney cell lines (ATCC, Manassas, Va.) were grown according to the distributor's instructions and infected according to standard protocols. For transfections using nanoplexes, A549 cells were seeded in 6-well plates to achieve 30-50% confluence (3.5×105 cells/well). 3 ug of RNA as GNR-RNA nanoplexes was added to each well in Opti-MEM. Efficiency of transfection was quantified using spectrophotometric measurements with excitation at 488 nm and emission at 510 nm from the lysed cells. At designated time points, cellular protein and RNA were harvested from duplicate wells for Western and qRT-PCR analyses. Total proteins were resolved on 4-15% SDS-PAGE gels, transferred to nitrocellulose membranes, and probed with commercial antibodies purchased from Sigma (actin) or Santa Cruz Biotechnology (RIG-I, MDA5, IPS1, and NS1). Quantitative RT-PCR (qRTPCR) was done with the SuperScript III Platinum SYBR Green One-Step kit (Invitrogen) in a Stratagene MX3000P thermal cycler according to the manufacturer's instructions. Primer sets used for these studies are as follows:

IFNβ:
forward 5′-TGG GAG GCT TGA ATA CTG CCT CAA-3′
reverse 5′-TCT CAT AGA TGG TCA ATG CGG CGT-3′
RIG-I:
forward 5′-AAA CCA GAG GCA GAG GAA GAG CAA-3′
reverse 5′-TCG TCC CAT GTC TGA AGG CGT AAA-3′
β-actin:
forward 5′-ACC AAC TGG GAC GAC ATG GAG AAA-3′
reverse 5′-TAG CAC AGC CTG GAT AGC AAC GTA-3′

PCR-Array data were collected using Interferon α, β Response PCR Array plates and analyzed using the RTç Profiler™ PCR Array Data Analysis software (SA Biosciences. Frederick, Md.). Quantification of secreted IFN-1 was performed using the Verikine Human IFN 1 ELISA Kit (PBL Interferon Source. Piscataway, N.J.) and the Synergy 4 plate reader (Biotek. Winooski, Vt.)

Statistical analysis: To determine the statistical significance between the 5′PPP-ssRNA, CIAPssRNA, or Capped-ssRNA treated and untreated groups, we used analysis of variance and a value of P<0.05 was considered significant. All data points were included in the analysis and there were no outliers. Studies of nanoplexes surface charge. GNR's were complexed with RNA's and zeta potential was acquired at 25° C. using a 90-Plus particle size analyzer (Brookhaven Instrument Corp., NY, USA).

Studies of Nanoplexes distribution in vitro: A459 cellular uptake of the nanoplexes (GNR-siRNAF), siPORT-siRNAF, free siRNAF distribution was monitored using dark-field and fluorescence microscopy. The siRNAF used in this study was purchased from Ambion (AM4620). The light-scattering images were recorded using an upright Nikon Eclipse 800 microscope with a high numerical dark-field condenser (NA 1.20-1.43, oil immersion) and a 100/1.4 NA oil Iris objective (Cfi Plan Fluor). In the dark-field configuration, the condenser delivers a narrow beam of white light from a tungsten lamp and the high NA oil immersion objective collects only the scattered light from the samples. Dark-field imaging was captured using a Q Imaging Micropublisher 3.3 RTV color camera. The Qcapture software was used for image acquisition. For fluorescence microscopy image, the upright Nikon Eclipse 800 microscope 100/1.4 NA oil Iris objective (Cfi Plan Fluor) was used and the Qlmaging Micropublisher 3.3 RTV color camera was used for image acquisition. (Ding, Yong et al. 2007). The signal from siRNAF was acquired using a filter 488 ex/510em, and for acquiring the signal from nuclear dye Hoechst a filter 405ex/460em was used.

Fluorescence Studies from A549 Cell Lysates: A459 cells were incubated with 50 pmols of free siRNAF, GNR-siRNAF, and siPORTsiRNAF nanoplexes and 24 hours later, cells were processed for fluorescence measurements. The medium was removed and the cells were lysed using M-PER (mammalian protein extraction reagent, Pierce Chemical Co.) and the PL spectrum was analyzed using a Horiba Jobin Yvon Fluorolog-3 spectrofluorometer.

MTT Cell Viability Assay: Viability of A459 cells was investigated up to 96 hours after treatment with GNR's complexes with RNA's. Cell viability assay measures the reduction of a tetrazolium component (3-(4,5-Dimethylthiazol-2-yl)-2,5-diphenyltetrazolium bromide, or MTT) into an insoluble formazan product by the mitochondria of viable cells. (Plumb 2004) Cells, in a 24-well plate (10,000 cells/well), were incubated with the MTT reagent for 3 hours, followed by addition of a detergent solution to lyse the cells and solubilize the colored crystals. The samples were read using an ELISA plate reader at 570 nm wavelength.

Agarose Gel Electrophoresis: GNR were complexed with 5′PPP-ssRNA and equivalent ssRNA that was free of 5′PPP (0.9 ug). The nanoplexes were added in individual wells in 1.5% agarose gel casted in Tris acetate-EDTA (TAE) buffer. (Bartlett, Su et al. 2007). The gel was run for 1.5 hours at 100 volts, stained with EtBr. Images of gel were obtained using an LM-20E UV benchtop transilluminator (UVP) in conjunction with an Olympus C-4000 zoom color digital camera with a UV filter.

Therapeutic Compositions

The nanoplex that delivers 5′PPP-ssRNA disclosed herein can be administered in vitro, ex vivo to a cell or subject. Generally, it is desirable to prepare the nanoplex as pharmaceutical compositions appropriate for the intended application. Accordingly, methods for making a medicament or pharmaceutical composition containing the polypeptides, nucleic acids, negative stranded RNA, single stranded RNA, double stranded RNA, siRNA, micro-RNA described above or included herein. Typically, preparation of a pharmaceutical composition (medicament) entails preparing a pharmaceutical composition that is essentially free of pyrogens, as well as any other impurities that could be harmful to humans or animals. Typically, the pharmaceutical composition contains appropriate salts and buffers to render the components of the composition stable and allow for uptake of nucleic acids or nanoplexes by target cells.

Therapeutic compositions can be provided as parenteral compositions, such as for injection or infusion. Such compositions are formulated generally by mixing a disclosed therapeutic agent at the desired degree of purity, in a unit dosage injectable form (solution, suspension, or emulsion), with a pharmaceutically acceptable carrier, for example one that is non-toxic to recipients at the dosages and concentrations employed and is compatible with other ingredients of the formulation. In addition, a disclosed therapeutic agent can be suspended in an aqueous carrier, for example, in an isotonic buffer solution at a pH of about 3.0 to about 8.0, preferably at a pH of about 3.5 to about 7.4, 3.5 to 6.0, or 3.5 to about 5.0. Useful buffers include sodium citrate-citric acid and sodium phosphate-phosphoric acid, and sodium acetate/acetic acid buffers. The active ingredient, optionally together with excipients, can also be in the form of a lyophilisate and can be made into a solution prior to parenteral administration by the addition of suitable solvents. Solutions such as those that are used, for example, for parenteral administration can also be used as infusion solutions.

Pharmaceutical compositions can include an effective amount of the nanoplex dispersed (for example, dissolved or suspended) in a pharmaceutically acceptable carrier or excipient. Pharmaceutically acceptable carriers and/or pharmaceutically acceptable excipients are known in the art and are described.

The nature of the carrier will depend on the particular mode of administration being employed. For example, parenteral formulations usually contain injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch or magnesium stearate. In addition, pharmaceutical compositions to be administered can contain minor amounts of non-toxic auxiliary substances, such as wetting or emulsifying agents, preservatives, and pH buffering agents and the like, for example sodium acetate or sorbitan monolaurate.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the pharmaceutical compositions is contemplated. Supplementary active ingredients also can be incorporated into the compositions. For example, certain pharmaceutical compositions can include the nanoplex in water, mixed with a suitable surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The pharmaceutical compositions (medicaments) can be prepared for use in prophylactic regimens (such as vaccines) and administered to human or non-human subjects (including birds, such as domestic fowl, for example, chickens, ducks, guinea fowl, turkeys and geese) to elicit an immune response against an influenza antigen (or a plurality of influenza antigens). Thus, the pharmaceutical compositions typically contain a pharmaceutically effective amount of the nanoplex.

In some cases the compositions are administered following infection to enhance the immune response, in such applications, the pharmaceutical composition is administered in a therapeutically effective amount. A therapeutically effective amount is a quantity of a composition used to achieve a desired effect in a subject. For instance, this can be the amount of the composition necessary to inhibit viral replication or to prevent or measurably alter outward symptoms of viral infection. When administered to a subject, a dosage will generally be used that will achieve target tissue concentrations (for example, in lymphocytes) that has been shown to achieve an in vitro or in vivo effect.

Administration of therapeutic compositions can be by any common route as long as the target tissue (typically, the respiratory tract) is available via that route. This includes oral, nasal, ocular, buccal, or other mucosal (such as rectal or vaginal) or topical administration. Alternatively, administration will be by orthotopic, intradermal subcutaneous, intramuscular, intraperitoneal, or intravenous injection routes. Such pharmaceutical compositions are usually administered as pharmaceutically acceptable compositions that include physiologically acceptable carriers, buffers or other excipients. In the case of transdermal delivery routes, such transdermal administration include but not be limited to patch, gel, foam, sponge, cream, spray, ointment or combinations thereof.

In some embodiments for administration of therapeutic compositions, any inhaler device may be used including but not limited to pressurized metered does inhalers, breath-activated inhalers, inhalers with spacer devices, nebulisers. In some embodiments for the transmucosal absorption administration, the administration may be accomplished by but is not limited to respiratory tract mucosal absorption, inhalation of vaporized, nebulized, powdered or aerosolized drug, as well as by direct instillation, oral transmucosal administration, sublingual administration, buccal administration, tablets, and nasal mucosal administration.

In various embodiments, the therapeutic compositions may be administered to the subject via any means including but not limited to gastrointestinal, enteral, central nervous system, epidural, intracerebral, intracerebroventricular, epicutaneous, intradermal, subcutaneous, nasal administration, intravenous, intraarterial, intramuscular, intracardiac, intraosseous infusion, intrasnovial, intrathecal, intraperitoneal, intravesical, intravitreal, intracavernous injection, intravaginal, intrauterine, transdermal, transmucosal, topical, epicutaneous, inhalational, enema, eye drops, ear drops, through mucous membranes, enteral, by mouth, by gastric feeding tube, by duodenal feeding tube, by gastronomy, rectally, pulmonary, buccal, ophthalmic, by bolus injection, via suppository drugs, intravenously, intra-arterial, intraosseous infusion, intra-muscular, inhalation, pill form, syrup, injection, by catheter, in dosage form, by drug injection, gas jet driven non-needle injection, intra-muscular needle injection, by hypodermic needle, by medical injection.

The pharmaceutical compositions can also be administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain about 100 mg of human serum albumin per milliliter of phosphate buffered saline. Other pharmaceutically acceptable carriers include aqueous solutions, non-toxic excipients, including salts, preservatives, buffers and the like may be used. Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components of the pharmaceutical composition are adjusted according to well-known parameters.

Additional formulations are suitable for oral administration. Oral formulations can include excipients such as, pharmaceutical grades of mannitol, lactose, starch, magnesium stearate, sodium saccharine, cellulose, magnesium carbonate and the like. The compositions (medicaments) typically take the form of solutions, suspensions, aerosols or powders.

In some embodiments, the pharmaceutical compositions disclosed herein may be delivered via oral administration to a subject, and as such, these compositions may be formulated with an inert diluent or with an assailable edible carrier, or they may be enclosed in hard- or soft-shell gelatin capsule, or they may be compressed into tablets, or they may be incorporated directly with the food of the diet.

The active compounds may even be incorporated with excipients and used in the form of ingestible tablets, buccal tables, troches, capsules, elixirs, suspensions, syrups, wafers, and the like. The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present invention may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as those containing sodium borate, glycerin and potassium bicarbonate, or dispersed in a dentifrice, including: gels, pastes, powders and slurries, or added in a therapeutically effective amount to a paste dentifrice that may include water, binders, abrasives, flavoring agents, foaming agents, and humectants, or alternatively fashioned into a tablet or solution form that may be placed under the tongue or otherwise dissolved in the mouth. When the route is topical, the form may be a cream, ointment, salve or spray. Also, adhesive bandages could be used for the administration of vaccines.

In some embodiments, the administration of agonist pharmaceutical compositions by intranasal sprays, inhalation, and/or other aerosol delivery vehicles is also considered. Following formation, the nanoplex is made into a solution or suspension for aerosolization, using a pharmaceutically acceptable excipient. Suitable excipients will be those that neither cause irritation to the pulmonary tissues nor significantly disturb ciliary function. Excipients such as water, aqueous saline (with or without buffer), dextrose and water, or other known substances, can be employed with the subject invention. The exact concentration and volume of the solution are not critical, acceptable formulations being readily determined by those of ordinary skill in the art. The concentration and volume of the solution will generally be dictated by the particular nebulizer selected to deliver the complex, and, the intended dose. It is preferred to minimize the total volume, however, to prevent unduly long inhalation times for the subject.

In some methods for delivering the nanoplex therapeutic composition directly to the lungs, the nanoplex is aerosolized by any appropriate method. Usually, the aerosol will be generated by a medical nebulizer system which delivers the aerosol through a mouthpiece, facemask, etc. from which the subject can draw the aerosol into the lungs. Various nebulizers are known in the art and can be used in the method of the present invention. The selection of a nebulizer system will depend on whether alveolar or airway delivery (e.g., trachea, pharynx, bronchi, etc.), is desired. Examples of nebulizers useful for alveolar delivery include but are not limited to the Acorn 1 nebulizer, and the Respirgard II® Nebulizer System, both available commercially from Marquest Medical Products, Inc., Inglewood, Colo. Other commercially available nebulizers for use with the instant invention include the UltraVent®. nebulizer available from Mallinckrodt, Inc. (Maryland Heights, Mo.); the Wright nebulizer (Wright, B. M., Lancet (1958) 3:24-25); and the DeVilbiss nebulizer (Mercer et al., Am. Ind. Hyg. Assoc. J. (1968) 29:66-78; T. T. Mercer, Chest (1981) 80:6(Sup) 813-817). Nebulizers useful for airway delivery include those typically used in the treatment of asthma. Such nebulizers are also commercially available.

Methods for delivering nanoplexes and other therapeutic compositions directly to the lungs via nasal aerosol sprays, and delivery of drugs using intranasal microparticle resins (Takenaga et al., 1998) and lysophosphatidyl-glycerol compounds are also well-known in the pharmaceutical arts and are proper methods of delivery. Likewise, transmucosal drug delivery in the form of a polytetrafluoroethylene support matrix is a proper method of delivery.

In one embodiment the transmucosal drug delivery device is in the form of a sheet material. The device contains an acid-containing particulate polymeric resin dispersed throughout a polytetrafluoroethylene support matrix. There is a flexible film backing on one side of the device. The backing is preferably a flexible film that prevents bulk fluid flow and is inert to the ingredients of the device. The backing protects the composition from excessive swelling and loss of adhesion over the time period during which the composition is intended to remain adhered to the mucosal surface. In the case of a device that contains a drug intended to be delivered to or across a mucosal surface (as opposed to delivery to the vicinity of the mucosal surface, e.g., to the oral cavity), the film backing material is preferably substantially impermeable to the drug and therefore it effectively prevents migration of the drug out of the coated portion of the device. In the case of a device that contains a drug intended to be delivered, e.g., to the oral cavity or the vaginal cavity, the backing can be permeable to the agent to be delivered and can be permeable to saliva as well.

The backing can be any of the conventional materials used as backing for tapes or dressings, such as polyethylene, polypropylene, ethylene-vinyl acetate copolymer, ethylene propylene diene copolymer, polyurethane, rayon, and the like. Non-woven materials such as polyesters, polyolefins, and polyamides can also be used. Also, a layer of a hydrophobic elastomer such as polyisobutylene can function as a backing. Preferred backing materials include an acrylate pressure-sensitive adhesive coated polyurethane film such as TEGADERM™ brand surgical dressing (commercially available from the 3M Company, St. Paul, Minn.).

The most preferred flexible film backings occlude substantially all of the surface area of the patch other than that surface that is intended to be adhered to the mucosal surface, while the surface of the patch that is to be adhered to the mucosal surface is substantially free of the backing. When the device is in use there is substantially no uncoated surface area of the device (such as uncoated sides or edges) exposed to mucus into which the drug can be delivered inadvertently.

The most preferred backing materials are also substantially insoluble in mucus and other fluids endogenous to the mucosal surface (e.g., in a device intended to adhere to buccal mucosa or other oral mucosa the backing is substantially insoluble in saliva). “Substantially insoluble” as used herein means that a thin coating (e.g., 0.1 mm thick) of the film backing material will not be eroded such that areas become exposed when a device is in place on a mucosal surface for a period of several hours.

The most preferred film backing materials include those that can be taken up in solution or suspension and applied (e.g., by brushing, spraying, or the like) from solution or suspension, and those that can be applied in the form of liquid prepolymeric systems and subsequently cured. These preferred film backing materials include polymeric materials and polymeric systems that are commonly used as enteric coatings or controlled release coatings. Exemplary materials include cellulose derivatives (e.g., ethylcellulose, cellulose acetate butyrate, cellulose acetate, cellulose acetate phthalate, hydroxypropyl methylcellulose phthalate, chitin, chitosan), polyvinyl alcohol and derivatives thereof such as polyvinyl acetate phthalate, shellac, zein, silicone elastomers, and polymethacrylates (e.g., cationic polymers based on dimethylaminoethyl methacrylate such as those copolymers available as EUDRAGIT™ type E, L, and S copolymers, copolymers of acrylic and methacrylic acid esters containing quaternary ammonium groups such as those copolymers available as EUDRAGIT™ type RS and RL copolymers, and others known to those skilled in the art). Most preferred backing materials include zein and ethylcellulose.

A device can contain other ingredients, for example excipients such as flavorings or flavor-masking agents, dyes, penetration enhancers, water-soluble or water-swellable fibrous reinforcers, and the like under circumstances and in amounts easily determined by those skilled in the art. Penetration enhancers have particular utility when used with drugs such as peptides and proteins. Suitable penetration enhancers include anionic surfactants (e.g., sodium lauryl sulfate); cationic surfactants (e.g., cetylpyridinium chloride); nonionic surfactants (e.g., polysorbate 80, polyoxyethylene 9-lauryl ether, glyceryl monolaurate); lipids (e.g., oleic acid); bile salts (e.g., sodium glycocholate, sodium taurocholate); and related compounds (e.g., sodium tauro-24,25-dihydrofusidate).

In some embodiments, the pharmaceutical compositions disclosed herein may be administered parenterally, intravenously, intramuscularly, or even intraperitoneally. Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

Typically, these formulations may contain at least about 0.1% of the active compound or more, although the percentage of the active ingredient(s) may, of course, be varied and may conveniently be between about 1 or 2% and about 60% or 70% or more of the weight or volume of the total formulation. Naturally, the amount of active compound(s) in each therapeutically useful composition may be prepared in such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

The pharmaceutical forms suitable for injectable use include sterile aqueous solutions or dispersions and sterile powders for the extemporaneous preparation of sterile injectable solutions or dispersions In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial ad antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In one embodiment an injectable particle can be prepared that includes a substance to be delivered and a polymer that is bound to a biologically active molecule, wherein the particle is prepared in such a manner that the biologically active molecule is on the outside surface of the particle. Injectable particles with antibody or antibody fragments on their surfaces can be used to target specific cells or organs as desired for the selective dosing of drugs a wide range of biologically active materials or drugs can be incorporated into the polymer at the time of nanoparticle formation. The substances to be incorporated should not chemically interact with the polymer during fabrication, or during the release process. Additives such as inorganic salts, BSA (bovine serum albumin), and inert organic compounds can be used to alter the profile of substance release, as known to those skilled in the art. Biologically-labile materials, for example, procaryotic or eucaryotic cells, such as bacteria, yeast, or mammalian cells, including human cells, or components thereof, such as cell walls, or conjugates of cellular can also be included in the particle. The term biologically active material refers to a peptide, protein, carbohydrate, nucleic acid, lipid, polysacccaride or combinations thereof, or synthetic inorganic or organic molecule, that causes a biological effect when administered in vivo to an animal, including but not limited to birds and mammals, including humans. Nonlimiting examples are antigens, enzymes, hormones, receptors, and peptides. Examples of other molecules that can be incorporated include nucleosides, nucleotides, antisense, vitamins, minerals, and steroids.

The period of time of release, and kinetics of release, of the substance from the nanoparticle will vary depending on the copolymer or copolymer mixture or blend selected to fabricate the nanoparticle. Given the disclosure herein, those of ordinary skill in this art will be able to select the appropriate polymer or combination of polymers to achieve a desired effect.

In one embodiment the device may be a single or multiple daily subcutaneous injection of nanoplex. Several other methods delivery are now available or in development, including (a) continuous subcutaneous nanoplex infusion by a wearable infusion pump; (c) implantation of a programmable nanoplex pump; (d) oral, nasal, rectal and transdermal mechanisms of nanoplex delivery; (e) administration of nanoplex analogues; (f) implantation of polymeric capsules which give continuous or time-pulsed release of nanoplex.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

Optionally, the pharmaceutical compositions or medicaments can include a suitable adjuvant to increase the immune response. As used herein, an “adjuvant” is any potentiator or enhancer of an immune response. The term “suitable” is meant to include any substance which can be used in combination with the nanoplex to augment the immune response, without producing adverse reactions in the vaccinated subject. Effective amounts of a specific adjuvant may be readily determined so as to optimize the potentiation effect of the adjuvant on the immune response of a vaccinated subject. For example, 0.5%-5% aluminum hydroxide (or aluminum phosphate) and MF-59 oil emulsion (0.5% polysorbate 80 and 0.5% sorbitan trioleate. Squalene (5.0%) aqueous emulsion) are adjuvants which have been favorably utilized in the context of influenza vaccines. Other adjuvants include mineral, vegetable or fish oil with water emulsions, incomplete Freund's adjuvant, E. coli J5, dextran sulfate, iron oxide, sodium alginate, BactoAdjuvant, certain synthetic polymers such as Carbopol (BF Goodrich Company, Cleveland, Ohio), poly-amino acids and co-polymers of amino acids, saponin, carrageenan, REGRESSIN™ (Vetrepharm, Athens, Ga.), AVRIDINE (N,N-dioctadecyl-N′,N′-bis(2-hydroxyethyl)-propanediamine), long chain poly dispersed (3 (1,4) linked mannan polymers interspersed with O-acetylated groups (for example ACEMANNAN), deproteinized highly purified cell wall extracts derived from a non-pathogenic strain of Mycobacterium species (for example EQUIMUNE®, Vetrepharm Research Inc., Athens Ga.), Mannite monooleate, paraffin oil, or muramyl dipeptide. A suitable adjuvant can be selected by one of ordinary skill in the art.

An effective amount of the pharmaceutical composition is determined based on the intended goal, for example vaccination of a human or non-human subject. The appropriate dose will vary depending on the characteristics of the subject, for example, whether the subject is a human or nonhuman, the age, weight, and other health considerations pertaining to the condition or status of the subject, the mode, route of administration, and number of doses, and whether the pharmaceutical composition includes nucleic acids or viruses. Generally, the pharmaceutical compositions described herein are administered for the purpose of stimulating or enhancing an immune response for example, an immune response against a viral antigen.

When administering a nanoplex, facilitators of nucleic acid uptake and/or expression can also be included, such as bupivacaine, carditoxin and sucrose, and transfection facilitating vehicles such as liposomal or lipid preparations that are routinely used to deliver nucleic acid molecules. Anionic and neutral liposomes are widely available and well known for delivering nucleic acid molecules. Cationic lipid preparations are also well known vehicles for use in delivery of nucleic acid molecules. Suitable lipid preparations include DOTMA (N-[1-(2,3-dioleyloxy)propyl]-N,N,N-trimethylammonium chloride), available under the tradename LIPOFECTIN®, and DOTAP (1,2-bis(oleyloxy)-3(trimethylammonio)propane). These cationic lipids may preferably be used in association with a neutral lipid, for example DOPE (dioleyl phosphatidylethanolamine). Still further transfection-facilitating compositions that can be added to the above lipid or liposome preparations include spermine derivatives and membrane-permeabilizing compounds such as GALA, Gramicidine S and cationic bile salts.

Alternatively, nucleic acids can be encapsulated, adsorbed to, or associated with, particulate carriers. Suitable particulate carriers include those derived from polymethyl methacrylate polymers, as well as PLG microparticles derived from poly (lactides) and poly (lactide-co-glycolides). Other particulate systems and polymers can also be used, for example, polymers such as polylysine, polyarginine, polyornithine, spermine, spermidine, as well as conjugates of these molecules.

A formulated vaccine composition can be created using our nanoplex with either an adenoviral vector and/or an adenovirus. An appropriate effective amount can be readily determined by one of skill in the art. Such an amount will fall in a relatively broad range that can be determined through routine trials, for example within a range of about 10 (xg to about 1 mg. However, doses above and below this range may also be found effective. The optimum carrier particle size will, of course, depend on the diameter of the target cells. Alternatively, colloidal gold particles can be complexed with our nanoplexes wherein the coated colloidal gold is administered (for example, injected) into tissue (for example, skin or muscle) and subsequently taken-up by immune-competent cells.

Tungsten, gold, platinum and iridium carrier particles can be used in conjunction with our nanoplexes. Tungsten and gold particles are preferred. Tungsten particles are readily available in average sizes of 0.5 to 2.0 um in diameter. Although such particles have optimal density for use in particle acceleration delivery methods, and allow highly efficient coating with DNA, tungsten may potentially be toxic to certain cell types. Gold particles or microcrystalline gold (for example, gold powder A1570, available from Engelhard Corp., East Newark, N.J.) will also find use with the present methods. Gold particles provide uniformity in size and reduced toxicity.

A number of methods are known and have been described for coating or precipitating DNA or RNA onto gold or tungsten particles. Most such methods generally combine a predetermined amount of gold or tungsten with plasmid DNA, CaCl2 and spermidine. The resulting solution is vortexed continually during the coating procedure to ensure uniformity of the reaction mixture. After precipitation of the nucleic acid, the coated particles can be transferred to suitable membranes and allowed to dry prior to use, coated onto surfaces of a sample module or cassette, or loaded into a delivery cassette for use in a suitable particle delivery instrument, such as a gene gun. Alternatively, nucleic acid vaccines can be administered via a mucosal membrane or through the skin, for example, using a transdermal patch. Such patches can include wetting agents, chemical agents and other components that breach the integrity of the skin allowing passage of the nucleic acid into cells of the subject.

Therapeutic compositions that include a disclosed therapeutic agent can be delivered by way of a pump or by continuous subcutaneous infusions, for example, using a mini-pump. An intravenous bag solution can also be employed. One factor in selecting an appropriate dose is the result obtained, as measured by the methods disclosed here, as are deemed appropriate by the practitioner. Other controlled release systems are discussed in Langer (Science 249:1527-33, 1990).

In one example, a pump is implanted. Implantable drug infusion devices are used to provide patients with a constant and long-term dosage or infusion of a therapeutic agent. Such device can be categorized as either active or passive. For example, in one embodiment the device is an implantable device and osmotic pump and catheter systems for delivering a pharmaceutical agent to a patient at selectable rates include an impermeable pump housing and a moveable partition disposed within the housing, the partition dividing the housing into an osmotic driving compartment having an open end and a pharmaceutical agent compartment having a delivery orifice. A plurality of semi permeable membranes may be disposed in the open end of the osmotic driving compartment and a number of impermeable barriers may seal selected ones of the plurality of semi permeable membranes from the patient until breached. Breaching one or more of the impermeable barriers increases the surface area of semi permeable membrane exposed to the patient and controllably increases the delivery rate of the pharmaceutical agent through the delivery orifice and catheter. Each of the plurality of semi permeable membranes may have a selected surface area, composition and/or thickness, to allow a fine-grained control over the infusion rate while the pump is implanted in the patient.

In another embodiment the device is an implantable drug infusion device which features a reliable and leak proof weld joint. The implantable drug infusion device features a hermetic enclosure; a drug reservoir positioned within the hermetic enclosure, a drug handling component, the drug handling component joined with a top surface of a docking station, the drug reservoir joined with a bottom surface of the docking station by a welded joint. The drug handling component typically being a MEMS-type device and fashioned from a silicon-glass or silicon-silicon sandwich. The docking station functions to isolate the thermal stresses created during the formation of the welded joint from the other joints and particularly from the joint between top surface of the docking station and the drug handling component. The thermal isolation function of the docking station is provided through one or more grooves within the docking station, the grooves functioning to separate, in a thermal manner, the top and bottom surfaces of the docking station.

Active drug or programmable infusion devices feature a pump or a metering system to deliver the agent into the patient's system. An example of such an active infusion device currently available is the Medtronic SYNCHROMED™ programmable pump. Passive infusion devices, in contrast, do not feature a pump, but rather rely upon a pressurized drug reservoir to deliver the agent of interest. An example of such a device includes the Medtronic ISOMED™.

In particular examples, therapeutic compositions including a disclosed therapeutic agent are administered by sustained-release systems. Suitable examples of sustainedrelease systems include suitable polymeric materials (such as, semi-permeable polymer matrices in the form of shaped articles, for example films, or microcapsules), suitable hydrophobic materials (for example as an emulsion in an acceptable oil) or ion exchange resins, and sparingly soluble derivatives (such as, for example, a sparingly soluble salt). Sustainedrelease compositions can be administered orally, parenterally, intracistemally, intraperitoneally, topically (as by powders, ointments, gels, drops or transdermal patch), or as an oral or nasal spray. Sustained-release matrices include polylactides copolymers of L-glutamic acid and gamma-ethyl-L-glutamate.

Polymers can be used for ion-controlled release. Various degradable and nondegradable polymeric matrices for use in controlled drug delivery are known in the art (Langer, Accounts Chem. Res. 26:537, 1993). For example, the block copolymer, polaxamer 407 exists as a viscous yet mobile liquid at low temperatures but forms a semisolid gel at body temperature. It has shown to be an effective vehicle for formulation and sustained delivery of recombinant interleukin-2 and urease (Johnston et al., Pharm. Res. 9:425, 1992; and Pec, J. Parent. Sci. Tech. 44(2):58, 1990). Alternatively, hydroxyapatite has been used as a microcarrier for controlled release of proteins (Ijntema et al., Int. J. Pharm. 112:215, 1994). In yet another aspect, liposomes are used for controlled release as well as drug targeting of the lipid-capsulated drug (Betageri et al., Liposome Drug Delivery Systems, Technomic Publishing Co., Inc., Lancaster, Pa., 1993). Numerous additional systems for controlled delivery of therapeutic proteins are known.

For example, in one embodiment the polymer may be a delivery system which is a solid but melts at body temperature. In particular the system is comprised of an emulsion which has been solidified by the use of such traditional components as the hard fats, waxes, fatty alcohols and acids and fatty acid esters. The systems contain at least 60% by volume and preferably 70% by volume of water or other nonlipoidal media. The systems may incorporate an active agent which is approved for or used for the treatment, prophylaxis, cure or mitigation of disease; for aesthetic or cosmetic usage; for diagnostic purposes; or for systemic drug therapy.

For example in one embodiment the polymer may be a new type of microsuspension and a method for its preparation. The microsuspension is formulated by suspending in an aqueous solution solid, water-insoluble microparticles, called lipospheres, that have a phospholipid layer embedded on their surface. The solid portion of the lipospheres can be either a solid substance to be delivered, or a substance dispersed in an inert solid vehicle, such as a wax. The lipospheres prepared as described herein are distinct from microdroplets or liposomes since the lipospheres have solid inner cores at room temperature and the phospholipid coating is entrapped and fixed to the particle surface.

The lipospheres are distinct from microspheres of uniformly dispersed material or homogenous polymer since they consist of at least two layers, the inner solid particle and the outer layer of phospholipid. The combination of solid inner core with phospholipid exterior confers several advantages to the lipospheres over microspheres and microparticles, including being highly dispersible in an aqueous medium, and having a release rate for the entrapped substance which is controlled by the phospholipid coating. There are also many advantages over other dispersion based delivery systems. Lipospheres have increased stability as compared to emulsion based delivery systems and are more effectively dispersed than most suspension based systems. Further, the substance to be delivered does not have to be soluble in the vehicle since it can be dispersed in the solid carrier. Further, in a liposphere, there is no equilibrium of substance in and out of the vehicle as in an emulsion system. Lipospheres also have a lower risk of reaction of substance to be delivered with vehicle than in emulsion systems because the vehicle is a solid inert material. Moreover, the release rate of the substance from the lipospheres can be manipulated by altering either or both the inner solid vehicle or the outer phospholipid layer.

Pharmaceutical uses of the lipospheres include in extended release injectable formulations; in oral formulations for release into the lower portions of the gastrointestinal tract; in oral formulations to mask the taste or odor of the substance to be delivered; and as components in lotions and sprays for topical use, for example, in dermal, inhalation, and cosmetic preparation

Another embodiment may be a method for encapsulating biologically active materials in synthetic, oligolamellar lipid vesicles (liposomes). The method comprises providing a mixture of lipid in organic solvent and an aqueous mixture of the material for encapsulation, emulsifying the provided mixture, removing the organic solvent and suspending the resultant gel in water. The method of the invention is advantageous over prior art methods of encapsulating biologically active materials in that it provides a means for a relatively high capture efficiency of the material for encapsulation. The disclosure is also of intermediate compositions in the encapsulation method, the product vesicles, compositions including the product vesicles as an active ingredient and their use.

One embodiment is a biochemical membrane covered with sialic residues thereby provides a coating that masks the surface membrane from recognition and removal by the scavenging RES cells of the body. The embodiment may be synthesized by constructing a biochemical membrane that is covered with sialic acid residues. These sialic acid residues provide a unique coating that masks the surface of the membrane from recognition by the scavenging cells of the body thereby allowing the membrane to survive and circulate systemically for an indefinite period of time. For drug delivery purposes, it is necessary that the membrane envelop an interior aqueous core volume so that it is capable of entrapping drugs and pharmaceutical agents. The vesicle has a chemical composition resulting from sialic acid residues on exterior surfaces of the membrane that differs significantly from the composition of the traditional array of drug carrier systems. Thus, the vesicle not only has a totally different chemical composition which results in new and unique properties, but also is capable of performing different and specialized functions in biological systems. One example of this function is the evasion of the scavenging cells of the body so as to permit it to circulate throughout the system.

One embodiment is a delivery system which is a solid but melts at body temperature. In particular the system is comprised of an emulsion which has been solidified by the use of such traditional components as the hard fats, waxes, fatty alcohols and acids and fatty acid esters. The systems contain at least 60% by volume and preferably 70% by volume of water or other nonlipoidal media. The systems may incorporate an active agent which is approved for or used for the treatment, prophylaxis, cure or mitigation of disease; for aesthetic or cosmetic usage; for diagnostic purposes; or for systemic drug therapy.

One embodiment is a peptide in an oil-in-water type submicron emulsion (SME) in which the mean particle size is in the range of 10 to 600 nm, more preferably 30 to 500 nm, commonly 50-300 nm. These formulations are suitable for administration by oral or rectal, vaginal, nasal, or other mucosal surface route. Moreover, bioadhesive polymers such as those currently used in pharmaceutical preparations optionally may be added to the emulsion to further enhance the absorption through mucous membranes. Bioadhesive polymers optionally may be present in the emulsion. Use of bioadhesive polymers in pharmaceutical emulsions affords enhanced delivery of peptides in bioadhesive polymer-coated suspensions. Bioadhesive pharmaceutical emulsions: a) prolong the residence time in situ, thereby decreasing the number of peptide drug administrations required per day; and b) may be localized in the specified region to improve and enhance targeting and bioavailability of delivered peptides.

In one embodiment the polypeptides called receptor mediated permeabilizers (RMP) may be used, which, increase the permeability of the blood-brain barrier to molecules such as therapeutic agents or diagnostic agents. These receptor mediated permeabilizer A-7 or conformational analogues can be intravenously co-administered to a host together with molecules whose desired destination is the cerebrospinal fluid compartment of the brain. The permeabilizer A-7 or conformational analogues allow these molecules to penetrate the blood-brain barrier and arrive at this destination

In one embodiment the chimeric peptides may be used in delivering a wide variety of neuropharmaceutical siRNA agents to the brain. The invention is particularly well suited for delivering neuropharmaceutical agents which are hydrophilic peptides. These hydrophilic peptides are generally not transported across the blood-brain barrier to any significant degree. Exemplary hydrophilic peptide neuropharmaceutical agents are: thyrotropin releasing hormone (TRH)—used to treat spinal cord injury and Lou Gehrig's disease; vasopressin—used to treat amnesia; alpha interferon—used to treat multiple sclerosis; somatostatin—used to treat Alzheimer's disease; endorphin—used to treat pain; L-methionyl (sulfone)-L-glutamyl-L-histidyl-L-phenylalanyl-D-lysyl-L-phenylalanine (an analogue of adrenocorticotrophic hormone (ACTH)-4-9)—used to treat epilepsy; and muramyl dipeptide—used to treat insomnia. All of these neuropharmaceutical peptides are available commercially or they may be isolated from natural sources by well-known techniques.

In one embodiment Protein microspheres are formed by phase separation in a non-solvent followed by solvent removal. The preferred proteins are prolamines, such as zein, that are hydrophobic, biodegradable, and can be modified proteolytically or chemically to endow them with desirable properties, such as a selected degradation rate. Composite microspheres can be prepared from a mixture of proteins or a mixture of proteins with one or more bioerodible polymeric materials, such as polylactides. Protein coatings can also be made. Compounds are readily incorporated into the microspheres for subsequent release. The process does not involve agents which degrade most labile proteins. The microspheres have a range of sizes and multiple applications, including drug delivery and delayed release of pesticides, fertilizers, and agents for environmental cleanup. Selection of microsphere size in the range of less than five microns and mode of administration can be used to target the microparticles to the cells of the reticuloendothelial system, or to the mucosal membranes of the mouth or gastrointestinal tract. Larger implants formed from the microspheres can also be utilized.

Treatable Viruses and Diseases

This disclosure relates to methods for inhibiting a viral infection in a subject. These methods include selecting a subject in whom the viral infection is to be inhibited and administering an effective amount of the disclosed negative stranded RNA, nanoparticles, and nanoplexes to a subject, thereby inhibiting the viral infection in the subject. In some embodiments, the viral infection is from a RNA virus, for example a ds RNA virus or a ssRNA virus. In some embodiments, the viral infection is a positive sense ssRNA virus. In other embodiments, the ssRNA virus is a negative sense RNA virus. In some embodiments the ssRNA viral infection is an influenza infection, such as an infection from influenza A, influenza B, a pandemic strain and or avian strain of influenza. In specific examples, the influenza infection is an infection with influenza strain H5N1, strain H7N7, or strain H9N2.

In some embodiments the viral infection is a virus or any viral variant including but not limited to Influenza A viruses, Influenza B viruses, Influenza C viruses, any Influenza viruses, hantaviruses, Lassa virus, rabies virus, Ebola virus, Marburg virus, measles virus, canine distemper virus, rinderpest virus, respiratory syncytial virus (RSV), mumps virus, human parainfluenza virus type 1, human parainfluenza virus type 2, human parainfluenza virus type 3, human parainfluenza virus type 4, Nipah virus, paramyxovirus, rubulavirus, morbillivirus, H1N1 virus also known as swine flu, H5N1 also known as avian flu, HPV, Hepatitis Virus, Crimean-Congo hemorrhagic fever, Human Immunodeficiency Virus (HIV), Human T-Lymphotropic Virus Type 1, Hepatitis B Virus, Epstein-Barr Virus, Cytomegalovirus, Herpes Simplex Virus bacterial viruses, bunyaviruses, arenaviruses, and any pandemic virus.

In some embodiments the viral infection is of the viral order including but not limited to Mononegavirales. In some embodiments the viral infection is of the viral family including but not limited to Bornaviridae, Filoviridae, Paramyxoviridae, Rhabdoviridae, Arenaviridae, Bunyaviridae, Orthomyxoviridae. In other embodiments the viral infection is of the viral Genus including but not limited to Deltavirus, Nyavirus, Ophiovirus, Tenuivirus, and Varicosavirus.

In some embodiments, a subject who already has a viral infection is selected for administration of an effective amount of the disclosed nanoplex. In other embodiments, a subject who does not yet have a viral infection is selected for administration of an effective amount of the disclosed nanoplex. For example, the subject has been exposed to a virus that may result in a viral infection in the subject.

F. Nanoparticles for use in the Nanoplex Composition

In various embodiments, the nanoplex particle can consist of any nanoparticle, nanoparticulate or nanocrystal of any shape, size or form including but not limited to a polymer, lipid, dendrimer, dendrimer-type polymer, branch-type polymer, decomposable polymer, dendrimer-type structure, carbon nanotube, ceramic nanoparticle, nanosphere, metal nanoshell, quantum dot, nanorod, nanocrystal, liposome nanoparticle, iron oxide nanoparticle, polymeric nanoparticle, fullerene, liquid crystal, supermagnetic nanoparticle, colloid, nanopowder, nanocup, nanosphere, nanodiamond, nanostar, nanowire, plasmid and other nanoparticles, including those nanoparticles that possess a cationic or anionic charge.

Ouantum Dot: In one embodiment, the nanoparticle component of the nanoplex can be a luminescent semiconductor nanocrystal compound comprised of a semiconductor nanocrystal capable of luminescence and/or absorption and/or scattering or diffraction when excited by an electromagnetic radiation source (of broad or narrow bandwidth) or a particle beam, and capable of exhibiting a detectable change in absorption and/or of emitting radiation in a narrow wavelength band and/or scattering or diffracting when excited. The semiconductor compound can be an element which includes but is not limited to Group II-IV semiconductor, Group III-V semiconductor, or MgS, MgSe, MgTe, CaS, CaSe, CaTe, SrS, SrSe, SrTe, BaS, BaSe, BaTe, ZnS, ZnSe, ZnTe, CdS, CdSe, CdTe, HgS, HgSe, or HgTe.

Metal Nanospheres and Metal nanoshells: In one embodiment, the nanoparticle component of the nanoplex can be a nanoparticle, wherein the nanoparticle is a material including but not limited to any noble metal, cadmium selenide, titanium, titanium dioxide, tin, tin oxide, silicon, silicon dioxide iron, iron̂III, oxide, silver, nickel, gold, copper, aluminum, steel, cobalt-chrome alloy, titanium alloy, brushite, tricalcium phosphate, alumina, silica, zirconia, diamond, polystyrene, silicone rubber, polycarbonate, polyurethanes, polypropylenes, polymethylmethacrylate, polyvinyl chloride, polyesters, polyethers, or polyethylene.

Silver Nanoparticles: In embodiments, the silver-containing nanoparticles are composed of elemental silver or a silver composite. Besides silver, the silver composite may include either or both of (i) one or more other metals and (ii) one or more non-metals. Suitable other metals include, for example, Al, Au, Pt, Pd, Cu, Co, Cr, In, and Ni, particularly the transition metals, for example, Au, Pt, Pd, Cu, Cr, Ni, and mixtures thereof. Exemplary metal composites are Au—Ag, Ag—Cu, Au—Ag—Cu, and Au—Ag—Pd. Suitable non-metals in the metal composite include, for example, Si, C, and Ge. The various components of the silver composite may be present in an amount ranging for example from about 0.01% to about 99.9% by weight, particularly from about 10% to about 90% by weight. In embodiments, the silver composite is a metal alloy composed of silver and one, two or more other metals, with silver comprising, for example, at least about 20% of the nanoparticles by weight, particularly greater than about 50% of the nanoparticles by weight. Unless otherwise noted, the weight percentages recited herein for the components of the silvercontaining nanoparticles do not include the stabilizer, that is, initial stabilizer and/or replacement stabilizer.

The initial stabilizer on the surface of the silver-containing nanoparticles can be any suitable compound such as a compound comprising a moiety selected from the group consisting of —NH2 such as butylamine, pentylamine, hexylamine, heptylamine, octylamine, nonylamine, decylamine, undecylamine, dodecylamine, tridecylamine, tetradecylamine, pentadecylamine, hexadecylamine, oleylamine, octadecylamine, diaminopentane, diaminohexane, diaminoheptane, diaminooctane, diaminononane, diaminode cane, diaminooctane, —NH— such as dipropylamine, dibutylamine, dipentylamine, dihexylamine, diheptylamine, dioctylamine, dinonylamine, didecylamine, methylpropylamine, ethylpropylamine, propylbutylamine, ethylbutylamine, ethylpentylamine, propylpentylamine, butylpentylamine, polyethyleneimine, an ammonium salt such as tributylammonium bromide, didodecyldimethylammonium bromide, benzyltriethylammonium chloride, —SH such as butanethiol, pentanethiol, hexanethiol, heptanethiol, octanethiol, nonanethiol, decanethiol, undecanethiol, dodecanethiol, —OC(═S)SH (xanthic acid), such as O-methylxanthate, O-ethylxanthate, O-propylxanthic acid, O-butylxanthic acid, O-pentylxanthic acid, O-hexylxanthic acid, O-heptylxanthic acid, O-octylxanthic acid, O-nonylxanthic acid, O-decylxanthic acid, O-undecylxanthic acid, O-dodecylxanthic acid, —S02M (M is Li, Na, K, or Cs) such as sodium octylsulfate, sodium dodecylsulfate, —OH (alcohol) such as terpinol, starch, glucose, poly(vinyl alcohol), —C5H4N (pyridyl) such as poly(vinylpyridine), poly(vinylpyridine-co-styrene), poly(vinylpyridine-co-butyl methacrylate), —COOH such as butyric acid, pentanoic acid, hexanoic acid, heptanoic acid, octanoic acid, nonanoic acid, decanoic acid, undecanoic acid, dodecanoic acid, tridecanoic acid, myristic acid, pentadecanoic acid, palmitic acid, heptadecanoic acid, stearic acid, oleic acid, nonadecanoic acid, icosanoic acid, eicosenoic acid, elaidic acid, linoleic acid, palmitoleic acid, poly(acrylic acid), —COOM (M is Li, Na, or K) such as sodium oleate, elaidate, linoleate, palmitoleate, eicosenoate, stearate, polyacrylic acid, sodium salt), R′R″ P— and R′R″ P(=0)−(R′, R″, and R″ are independently an alkyl having for instance 1 to 15 carbon atoms or aryl having for instance 6 to 20 carbon atoms) such as trioctylphosphine and trioctylphosphine oxide, and the like, or a mixture thereof.

The carboxylic acid as the replacement stabilizer is different from the initial stabilizer and can be any suitable carboxylic acid such as, for example, butyric acid, pentanoic acid, hexanoic acid, heptanoic acid, octanoic acid, nonanoic acid, decanoic acid, undecanoic acid, dodecanoic acid, tridecanoic acid, myristic acid, pentadecanoic acid, palmitic acid, palmitoleic acid, heptadecanoic acid, stearic acid, oleic acid, elaidic acid, linoleic acid, nonadecanoic acid, icosanoic acid, eicosenoic acid, and the like, or a mixture thereof.

Carbon Nanotube: In one embodiment, the nanoparticle component of the nanoplex can be a nanoparticle that is a nanotube with a hollow tubular body defining an inner void, containing an open end on either side of the tube.

Ceramic Nanoparticles: In one embodiment, the nanoparticle component of the nanoplex can be of any ceramic material wherein one metal alkoxide or metal salt can be selected from but is not limited to Al, Ba, Mg, Ca, La, Fe, Si, Ti, Zr, Pb, Sn, Zn, Cd, As, Ga, Sr, Bi, Ta, Se, Te, Hf, Mg, Ni, Mn, Co, S, Ge, Li, B and Ce to be used as the ceramic material of the ceramic nanoparticle.

Nanocapsules: In one embodiment, the nanoparticle component of the nanoplex can be a nanometer-sized, hollow, spherically-shaped object that can be utilized to encapsulate small amounts of pharmaceuticals, enzymes, or other catalysts

Polymers: In one embodiment, the nanoparticle component of the nanoplex can be a polymer nanoparticle including but not limited to a synthetic polymers such as poly(ethylene glycol) (PEG), N-(2-hydroxylpropyl)methacrylamide (HPMA) co-polymers, poly(vinylpyrrolidone), poly(ethyleneimine), and linear polyamidoamines; natural polymers such as dextran, dextrin, hyaluronic acid, collagen, and chitosans; pseudosynthetic polymers such as poly(L-lysine), poly(L-glutamic acid), poly(malic acid), and poly(aspartamides). Of these polymers, PEG, HPMA, dextran, and poly(L-lysine) have been used repeatedly in the development of nanoparticle carriers. The structural architecture of the polymer can be but is not limited to a spherical, linear, branched, cross-linked, block, graft, multivalent, dendronized, or star-shaped structure.

Nanocompaite: In one embodiment, the nanoparticle component of the nanoplex can be a nanometer-scale composite structures composed of organic molecules intimately incorporated with inorganic molecules.

Nanowire: In one embodiment, the nanoparticle component of the nanoplex can be a nanometer-scale wire made of materials that conduct electricity. They can be coated with molecules such as antibodies that will bind to proteins and other substances.

Dendrimer: In one embodiment, the nanoparticle component of the nanoplex can be a biodegradable or non-biodegradable polymer defined by regular, highly branched monomers leading to a monodisperse, tree-like or generational structure with functional groups on the surface. The dendritic nanoparticle can vary by molecular weight and include but not be limited to dendronized polymers, hyperbranched polymers, a polymer brush. The dendrimer can be water soluble or non-water soluble.

Chitosan: In one embodiment the nanoparticle component of the nanoplex can be a chitosan particle. A chitosan particle is a linear polysaccharide composed of randomly distributed β-(1-4)-linked D-glucosamine (deacetylated unit) and N-acetyl-D-glucosamine (acetylated unit).

G. RNA Component of Use in the Nanoplex

In various embodiments, the nanoplex particle can consist of any negative stranded RNA also known as antisense-strand RNA, nucleotide sequence, or genetic material which stimulates a cellular signaling pathway directly or indirectly to express cytokines, chemokines and any other anti-viral, such genetic material includes but is not limited to 5′PPP-single stranded RNA, small interfering RNA, RNA interference, double stranded RNA molecules, and small interfering RNA.

What is claimed is:

1. A product comprising:
a nanoparticle bound to an siRNA nucleotide sequence or genetic material that produces siRNA

2. The product of claim 1 wherein the siRNA nucleotide sequence is 5′PPPNS1siRNA or NS1siRNA without 5′PPP moiety.

3. The product of claim 2 wherein the 5′PPPNS1siRNA binds to a helicase domain of RIG-I inducing type 1 interferon response.

4. The product of claim 1 wherein the siRNA nucleotide sequence is a NS1siRNA component that inhibits a key viral pathogenic factor.

5. The product of claim 1 wherein the siRNA nucleotide sequence is a NS1siRNA component that inhibits influenza.

6. The product of claim 1 wherein the siRNA nucleotide sequence is a siRNA component that inhibits negative and positive stranded RNA virus and DNA viral pathogenic factors.

7. The product of claim 1, wherein the nanoparticle is a quantum dot.

8. The product of claim 7, wherein the quantum dot does not contain a non-heavy metal core or outer shell.

9. The product of claim 1 configured to activate RIG-I and induce IFN-1 expression in A549 cells.

10. The product of claim 1 adapted to be delivered through an aerosolized inhaler.

11. The product of claim 1 adapted to be delivered intravenously.

12. The product of claim 1 adapted to be delivered orally.

13. A product comprising:
a nanoparticle bound to a micro RNA nucleotide sequence or genetic material.

14. The product of claim 13, wherein the nanoparticle is a quantum dot.

15. The product of claim 14, wherein the quantum dot is a non-heavy metal.

16. The product of claim 13 configured to activate RIG-I and induced IFN-1 expression in A549 cells.

17. A method for treating a patient, comprising:
delivering to the patient a set of a nanoparticles respectively bound to siRNA nucleotide sequence(s) or genetic material to activate RIG-I and induce IFN-1 expression.

18. The method of claim 17, wherein the delivery is accomplished through use of an aerosol.

19. The method of claim 18 wherein the delivery is accomplished through oral administration of the nanoparticles respectively bound to siRNA nucleotide sequence(s) or genetic material.
